(19) United States
(12) Patent Application Publication
(10) Pub. No.: US 2025/0018176 A1
(43) Pub. Date: Jan. 16, 2025

(54) ELECTRODE CONFIGURATIONS FOR ENDOVASCULAR THERAPY DEVICE

(71) Applicant: Covidien LP, Mansfield, MA (US)

(72) Inventors: Yao-Chuan Chang, New Brighton, MN (US); Haitao Zhang, Maple Grove, MN (US); Alan Shi, Plymouth, MN (US); Kevin V. Nguyen, Westminster, CA (US); Bernard Q. Li, Plymouth, MN (US)

(21) Appl. No.: 18/625,804

(22) Filed: Apr. 3, 2024

Related U.S. Application Data
(60) Provisional application No. 63/513,688, filed on Jul. 14, 2023.

(51) Int. Cl. A61N 1/05; A61N 1/36

(52) U.S. Cl. CPC A61N 1/05; A61N 1/36053

(57) ABSTRACT

In some examples, an endovascular medical device system includes an elongated body configured to be introduced in a blood vessel of a patient. The system includes an expandable structure at a distal portion of the elongated body. The expandable structure includes an expandable body portion including a plurality of interconnected struts. The expandable structure further includes a plurality of electrode attachment elements, where each electrode attachment element of the plurality of electrode attachment elements includes a first projection and a second projection branching off of at least one strut of the plurality of interconnected struts. In some examples, the system includes one or more electrodes coupled to the expandable structure via the plurality of electrode attachment elements. In some examples, each electrode of the one or more electrodes is configured to receive the first projection and the second projection to couple the respective electrode to the expandable structure.

This application claims the benefit of U.S. Provisional Application No. 63/513,688, filed Jul. 14, 2023, and entitled, “ENDOVASCULAR NERVE TISSUE STIMULATION THERAPY,” the entire content of which is incorporated herein by reference.

### TECHNICAL FIELD

This disclosure relates to electrical stimulation therapy.

### BACKGROUND

Medical devices, such as electrical stimulation devices, may be used in different therapeutic applications, such as vagus nerve stimulation (VNS) and/or deep brain stimulation (DBS). A medical device may be used to deliver therapy to a patient to treat a variety of symptoms or patient conditions. In some therapy systems, an external or an implantable electrical stimulator delivers electrical stimulation therapy to a target tissue site within a patient with the aid of one or more electrodes and/or senses one or more patient parameters with the aid of the one or more electrodes.

### SUMMARY

This disclosure describes example endovascular medical devices and systems configured to endovascularly deliver electrical stimulation therapy to a patient (e.g., to one or more nerves or brain targets) and/or sense one or more patient parameters (e.g., nerve signals, brain signals, and/or other physiological parameters), and related methods. In particular, this disclosure describes configurations for electrodes of endovascular medical devices and systems that facilitate delivery of electrical stimulation therapy and/or sensing patient parameters from an endovascular location.

In the examples described herein, an endovascular therapy system includes one or more electrodes that are carried by an expandable structure at a distal portion of an elongated body (e.g., a medical lead). The expandable structure is configured to transform between a delivery (e.g., compressed or relatively low-profile) configuration and a deployed (e.g., expanded) configuration. The expandable structure (e.g., a stent, or stent-like structure) includes struts and a plurality of electrode attachment elements. The electrode attachment elements facilitate mechanical coupling of the electrodes to the expandable structure. Thus, because electrodes can be fabricated separately from the expandable structure, electrode properties can be selected and/or adjusted during the electrode fabrication process for a particular end use, and the electrodes can subsequently be attached to the expandable structure via the electrode attachment elements.

Each electrode attachment element, which may include a portion of one or more struts of the expandable structure, include structural features (e.g., projections) to minimize or even prevent rotation of the electrodes with respect to the struts. Each electrode is configured to receive the features (e.g., projections) of respective electrode attachment elements to couple the respective electrode to the expandable structure.

Minimizing, or even preventing, rotation of electrodes with respect to struts can facilitate directional orientation of electrodes with respect to the expandable structure. For example, by minimizing, or even preventing, rotation of electrodes with respective to struts, a conductive surface of each electrode can be configured to face radially outward (relative to a central longitudinal axis of the expandable structure) from the expandable structure with little or no possibility of the conductive face being oriented radially inward, such as away from a blood vessel wall. In this way, the system is configured to facilitate directional electrical stimulation therapy and/or directional sensing, such as in a direction radially outward from the expandable structure and/or a blood vessel and towards a blood vessel wall.

As compared to other electrode designs (e.g., cylindrical electrodes) of the same mass, the electrode configurations described in this disclosure may include a relatively higher conductive surface area facing an anatomical region of interest (e.g., radially outward towards a vessel wall of a blood vessel). Said another way, the electrode configurations described in this disclosure include a relatively high surface area configured to face an anatomical region of interest without a relative increase in size and/or mass of an individual electrode. By fixing the particular surface of the electrode that faces radially outward towards a vessel wall, the systems described in this disclosure may be relatively more efficient (e.g., in terms of power used and/or power lost) during electrical stimulation therapy and/or sensing.

In some examples herein, electrodes are formed from a machined structure and/or thin film structures (e.g., such as in the case of stamped electrodes). Using thin film metallic structures for electrodes may enable individual electrodes to deform (e.g., flex) during delivery and deployment of the electrodes via the expandable structure. In some examples, the electrode attachment elements facilitate (e.g., accommodate) deformation or other movement of electrodes during delivery to a target location in the vasculature and deployment to a configuration in which electrodes are positioned in apposition with a vessel wall. For example, the electrode attachment elements described herein may allow electrodes to deform (e.g., elastically deform) around struts in one or more of the delivery or deployed configuration of the expandable structure (e.g., the stent or stent-like structure).

In some examples, an endovascular medical device system includes an elongated body configured to be introduced in a blood vessel of a patient and an expandable structure at a distal portion of the elongated body. In some examples, the expandable structure includes an expandable body portion including a plurality of interconnected struts and a plurality of electrode attachment elements, each electrode attachment element of the plurality of electrode attachment elements including a first projection and a second projection branching off of at least one strut of the plurality of interconnected struts. In some examples, the endovascular medical device system includes one or more electrodes coupled to the expandable structure via the plurality of electrode attachment elements, wherein each electrode of the one or more electrodes is configured to receive the first projection and the second projection of a respective electrode attachment element of the plurality of electrode attachment elements to couple the respective electrode to the expandable structure.

In some examples, a method of using a medical device system includes introducing a medical device into vasculature of a patient. In some examples, the medical device system includes an elongated body configured to be introduced in a blood vessel of a patient and an expandable structure at a distal portion of the elongated body. In some examples, the expandable structure includes an expandable body portion including a plurality interconnected struts and a plurality of electrode attachment elements, each electrode attachment element of the plurality of electrode attachment elements including a first projection and a second projection branching off of at least one strut of the plurality of interconnected struts. In some examples, the medical device system includes one or more electrodes coupled to the expandable structure via the plurality of electrode attachment elements, wherein each electrode of the one or more electrodes is configured to receive the first projection and the second projection of a respective electrode attachment element of the plurality of electrode attachment elements to couple the respective electrode to the expandable structure. In some examples, the method of using a medical device system includes advancing the medical device until the one or more electrodes are at or near a target location in the vasculature of the patient.

In some examples, an electrode includes an electrode body. In some examples, the electrode body defines a first fixation hole configured to receive a first projection of an expandable structure. In some examples, the electrode body defines a second fixation hole separate from the first fixation hole and configured to receive a second projection of the expandable structure. In some examples, the electrode body defines at least one conductor hole configured to receive a conductor wire, wherein the electrode body includes a first electrode surface, the first electrode surface being electrically conductive, wherein the electrode body includes a second electrode surface opposite the first electrode surface with an electrically insulative material applied to the second electrode surface, and wherein the electrode body defines a non-cylindrical shape.

The examples described herein may be combined in any permutation or combination.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example therapy system including an endovascular device configured to deliver electrical stimulation therapy to a target tissue site of a patient and/or sense a patient parameter from an endovascular location.

FIG. 2 is a functional block diagram illustrating components of an example medical device of the therapy system of FIG. 1.

FIG. 3 illustrates a distal portion of the example endovascular therapy system of FIG. 1 including an expandable structure and electrodes carried by the expandable structure.

FIG. 4A and FIG. 4B illustrate an example machined electrode configuration.

FIG. 4C illustrates an example pre-cut bulk electrode and a machined electrode.

FIGS. 5A and 5B illustrate an example stamped electrode configuration.

FIG. 5C illustrates an example pre-stamped sheet of conductive material and a stamped electrode.

FIG. 6 is a flow diagram illustrating an example technique for introducing and advancing an endovascular device according to this disclosure.

Like reference characters denote like elements throughout the description and figures.

### DETAILED DESCRIPTION

This disclosure describes devices, systems, and methods relating to delivery of electrical stimulation therapy, such as vagus nerve stimulation (VNS), deep brain stimulation (DBS), and/or sensing one or more patient parameters (e.g., nerve activity from one more nerves, cardiac signals, muscle activation signals, brain signals and/or other physiological parameters, such as impedance, electroencephalogram (EEG), evoked potentials, local field potentials, etc.) from an endovascular location. Example endovascular locations that can be used for electrical stimulation therapy (e.g., VNS therapy) and/or sensing using the devices described herein include an internal jugular vein (IJV). Example endovascular locations that can be used to access the brain sites for electrical stimulation therapy (e.g., DBS) and/or sensing using the devices described herein include any suitable cranial blood vessel (also referred to herein as a cerebral blood vessel or neurovasculature, which can include a vein or an cranial artery), such as, but not limited to, the thalamostriate vein, the internal cerebral vein, the basal vein of Rosenthal, the inferior sagittal sinus, the superior sagittal sinus, or the anterior choroidal artery.

VNS has been proposed for use to manage one or more patient conditions, such as to control an inflammatory response in patients. Stimulating the vagus nerve may dampen the inflammatory response and associated cytokine response. In some examples, inflammatory cytokines are modulated up or down via stimulation. In addition, VNS may assist in stroke rehabilitation and limit ischemia reperfusion injury. After a myocardial infarct or stroke, reperfusion therapies (surgery or drugs) are given to restore blood flow. However, due to the restoration of blood, flow induced local damage occurs, including ischemia reperfusion injury. This injury may induce local accumulations of chemical mediators such as reactive oxygen species (ROS) production, inflammatory cytokines, bradykinin, etc., which can further affect inflammation. Such inflammatory compounds may trigger sensory signaling, which can lead to a reduced organ vagus activity and sympathetic overdrive. Vagus nerve stimulation may treat reperfusion damage as the inflammatory state may be lowered by increasing parasympathetic drive.

DBS has been proposed for use to manage one or more patient conditions. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD). DBS can also reduce the symptoms of Parkinson's disease, dystonia, or cerebellar outflow tremor.

While this disclosure is primarily directed to examples of VNS and/or sensing via applicable endovascular locations (e.g., the internal jugular vein), it should be understood that the devices, systems, and techniques may be adapted for DBS, other kinds of brain stimulation, peripheral nerve stimulation, or electrical stimulation and/or sensing of any nerve tissue that can be done via an endovascular location.

In the examples described herein, an endovascular therapy system includes one or more electrodes that are carried by an expandable structure at a distal portion of an elongated body (e.g., a medical lead). The expandable structure is configured to transform between a delivery (e.g., compressed) configuration and a deployed (e.g., expanded) configuration. The expandable structure (e.g., a stent, or stent-like structure) includes struts and a plurality of electrode attachment elements. The electrode attachment elements facilitate mechanical coupling of the one or more electrodes to the expandable structure.

Because the electrodes described in this disclosure can be fabricated separately than the expandable structure, electrode properties can be selected and/or adjusted during the electrode fabrication process for a particular end use, and the electrodes can subsequently be attached to the expandable structure via the electrode attachment elements. Each electrode attachment element, which may include a portion of one or more struts of the expandable structure, include structural features (e.g., projections) to minimize or even prevent rotation of the electrodes with respect to the struts. Each electrode is configured to receive the features (e.g., projections) of respective electrode attachment elements to couple the respective electrode to the expandable structure.

Minimizing, or even preventing, rotation of electrodes with respect to struts can facilitate directional orientation of electrodes with respect to the expandable structure. For example, by minimizing, or even preventing, rotation of electrodes with respective to struts, a conductive surface of each electrode can be configured to face radially outward from the expandable structure (e.g., from a central longitudinal axis of the expandable structure) with little or no possibility of the conductive face being oriented radially inward, such as away from a blood vessel wall. In this way, the system is configured to facilitate directional electrical stimulation therapy and/or directional sensing, such as in a direction radially outward from the expandable structure and/or a blood vessel and towards a blood vessel wall.

As compared to other electrode designs (e.g., cylindrical electrodes) of the same or similar mass, the electrode configurations described in this disclosure may include a relatively higher conductive surface area facing an anatomical region of interest (e.g., radially outward towards a vessel wall of a blood vessel). Said another way, the electrode configurations described in this disclosure include a relatively high surface area facing an anatomical region of interest without a relative increase in size and/or mass of an individual electrode. By fixing the particular surface of the electrode that faces radially outward towards a vessel wall, the systems described in this disclosure may be relatively more efficient (e.g., in terms of power used and/or power lost) during electrical stimulation therapy and/or sensing.

In some examples herein, electrodes include features for coupling to expandable structures (e.g., stents or stent-like structures), as well as for coupling to conductor wires for electrically coupling the electrodes to a medical device. In some examples, the features enable the electrodes to receive portions (e.g., mating features, including projections) of the electrode attachment elements of the expandable structure to facilitate mechanical coupling of the electrodes to the expandable structure. Thus, the features of the electrodes can be considered complementary or mating structural features to the structural features of the electrode attachment elements. In some examples, electrodes are configured to receive and/or electrically couple to one or more conductor wires (e.g., for electrically coupling the electrode to a medical device). In some examples, electrodes include pass-through holes, such as for routing conductor wires to different electrodes.

The electrodes can be formed from any suitable material and using any suitable technique. In some examples herein, electrodes are formed from machined structures and/or thin film structures (e.g., such as in the case of stamped electrodes). Using thin film metallic structures for electrodes may enable individual electrodes to deform (e.g., flex) during delivery and deployment of the electrodes via the expandable structure. In some examples, the electrode attachment elements facilitate (e.g., accommodate) deformation or other movement of electrodes during delivery to a target location in the vasculature and deployment to a configuration in which electrodes are positioned in apposition with a vessel wall. For example, the electrode attachment elements described herein may allow electrodes to deform (e.g., elastically deform) around struts in one or more of the delivery or deployed configuration of the expandable structure (e.g., the stent or stent-like structure).

In some examples, a medical device is configured to generate electrical stimulation and/or sense a patient parameter via the electrodes of the endovascular device. The electrodes may be carried by or otherwise disposed on an expandable structure, which may be configured to orient the electrodes and/or anchor the electrodes at a particular location in the vasculature of the patient.

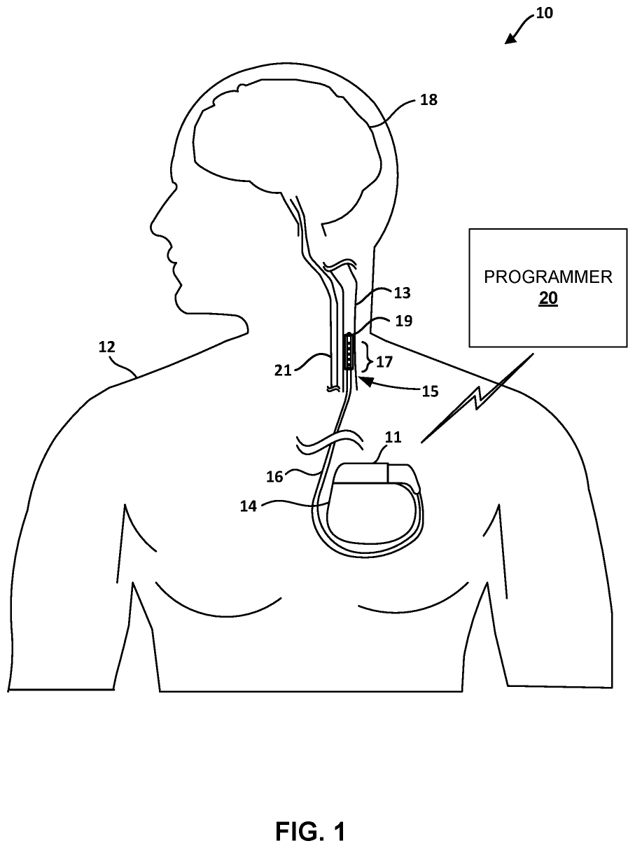

FIG. 1 is a conceptual diagram illustrating an example therapy system 10 configured to deliver electrical stimulation therapy to a target tissue site of a patient 12 or sense a patient parameter from an endovascular location. Patient 12 ordinarily will be a human patient. In some cases, however, therapy system 10 is applied to other mammalian or non-mammalian non-human patients. Therapy system 10 includes a medical device 14 and an endovascular device 16. In the example shown in FIG. 1, medical device 14 is configured to deliver electrical stimulation therapy (e.g., VNS) to a vagus nerve 21 of patient 12 and/or sense bioelectric signals via electrodes 17. However, in other examples, therapy system 10 and/or medical device 14 is configured to deliver electrical stimulation therapy (e.g., DBS) to brain 18 of patient 12 and/or sense bioelectrical brain signals in brain 18 via electrodes 17.

Endovascular device 16 is positioned in a jugular vein 13 of patient 12 such that one or more electrodes 17 are located proximate to a target tissue site. In particular, electrodes 17 are positioned to deliver electrical stimulation therapy to and/or sense signals from nerves surrounding jugular vein 13, including (but not limited to) vagus nerve 21. Endovascular device 16 includes an expandable structure 19 at a distal portion 15 of endovascular device 16 which may help hold electrodes 17 in apposition with a vessel wall (e.g., of jugular vein 13). In some examples, expandable structure 19 is at a distalmost end of endovascular device 16. Medical device 14 can provide electrical stimulation to one or more regions surrounding jugular vein 13 in order to manage a condition of patient 12, such as to mitigate the severity or duration of the patient condition.

Endovascular device 16 includes any suitable medical device configured to deliver electrical stimulation signals to tissue proximate electrodes 17. For example, endovascular device 16 can be a medical lead, a catheter, a guidewire, or another elongated body carrying electrodes 17 and configured to be electrically coupled to medical device 14 via an electrically conductive pathway that runs between medical device 14 and electrodes 17. Endovascular device 16 has any suitable length that enables connection to medical device 14 either directly or indirectly, e.g., a length of 150 centimeters (cm) to 250 cm, such as 200 cm. Further, endovascular device 16 has a suitable length (e.g., as measured along a longitudinal axis of endovascular device 16) for accessing a target tissue site within the patient from a vascular access point. In examples in which endovascular device 16 accesses the jugular vein 13 and/or vasculature in a brain 18 of patient 12 from a femoral artery access point at the groin of the patient, endovascular device 16 has a length of about 100 cm to about 200 cm, although other lengths may be used. As used herein, “about” may indicate the exact value or nearly the exact value to the extent permitted by manufacturing tolerances. “About” can also refer to a certain percentage of the recited value (e.g., within about 1%, 5%, or 10%).

Endovascular device 16 is configured to be introduced in the vasculature of patient 12, such as to access jugular vein 13 and/or relatively more distal locations in a patient, such as the middle cerebral artery (MCA) in a brain of a patient. Endovascular device 16 may include an elongated body that is structurally configured to be relatively flexible, pushable, and relatively kink-and buckle-resistant, so that it may resist buckling when a pushing force is applied to a relatively proximal portion to advance endovascular device 16 distally through vasculature, and so that it may resist kinking when traversing around a tight turn in the vasculature. Kinking and/or buckling of may hinder a clinician's efforts to push the elongated body distally, e.g., past a turn. In some examples, endovascular device 16 includes one or more radiopaque components (e.g., platinum bands) proximate electrodes 17 and/or expandable structure 19.

Instead of or in addition to the elongated body of endovascular device 16 being configured for intravascular navigation to a cerebral blood vessel to deliver electrical stimulation therapy or sense a patient parameter, endovascular device 16 can be navigated through vasculature (e.g., to jugular vein 13, brain 18, or other target tissue sites) with the aid of a guide member. The guide member can include an outer catheter, an inner catheter, a guide extension catheter, a guidewire, or the like or combination thereof.

In some examples, more than one endovascular device 16 is implanted within patient 12 to provide stimulation to and/or sense multiple anatomical regions, including one or more of both the left and right jugular veins, as well as in locations of brain 18. For example, two or more of endovascular device 16, which may be paired with one or more of medical device 14, may be configured of bilateral stimulation and/or sensing (e.g., of the left jugular vein and a right jugular vein). Endovascular device 16, including electrodes 17 and/or expandable structure 19, can be implanted in a blood vessel for chronic therapy delivery and/or chronic sensing (e.g., on the order of months or even years) or for more temporary therapy delivery and/or sensing (e.g., on the order of days, such as less than a month or less than 6 months). Temporary therapy delivery may include one or more trial periods, such as to determine, evaluate, or confirm an efficacy of stimulation and/or sensing.

The electrical stimulation therapy described herein (e.g., VNS) may be used to treat various patient conditions, such as, a variety of illnesses including, but not limited to: reperfusion damage, cardiac ischemia, brain ischemia, stroke, traumatic brain injury, surgical or non-surgical acute kidney injury, inability of the intestine (bowel) to contract normally and move waste out of the body, postoperative ileus, postoperative cognitive decline or postoperative delirium, asthma, sepsis, bleeding control, myocardial infarction reduction, dysmotility, and obesity. Treating any of these diseases may improve patient outcomes by shortening length of hospital stays and reducing medical costs.

The vasculature into which endovascular device 16 may be inserted and/or guided includes, but is not limited to, veins or arteries. For example, endovascular device 16 can be navigated from a vasculature access site (e.g., in the femoral artery, the radial artery, or another suitable access site) to one or more of a jugular vein (e.g., internal jugular vein and/or external jugular vein), a carotid artery (e.g., internal carotid artery, external carotid artery, and/or common carotid artery), as well as brain targets including the thalamostriate vein, the internal cerebral vein, the basal vein of Rosenthal, the inferior/superior sagittal sinus, the anterior choroidal artery, or any related combinations thereof.

A clinician can also select a particular blood vessel to position electrodes 17 within, such as to avoid certain regions to minimize or even eliminate adverse effects. For example, electrodes 17 can be oriented or positioned relative to vagus nerve 21 to avoid inadvertently providing electrical stimulation to anatomical regions (e.g., undesired anatomical regions) near the targeted anatomical region.

In some examples, endovascular device 16 is configured to be delivered to one or more target sites in vasculature of patient 12. Thus, rather than introducing endovascular device 16 into tissue in close proximity with vagus nerve 21 through an incision in the neck or chest area of patient 12, endovascular device 16 is configured to be navigated proximate to a target electrical stimulation site via vasculature of patient 12. The endovascular delivery of endovascular device 16 to target sites can help minimize the invasiveness of therapy system 10.

In some examples, electrodes 17 are positioned on (e.g., coupled to, defined by, or otherwise carried by) expandable structure 19 of endovascular device 16, which is configured to expand radially outwards from a relatively low-profile (e.g., radially compressed) delivery configuration to a deployed configuration. This may enable electrodes 17 to be held in apposition with a blood vessel wall, promote tissue ingrowth around electrodes 17 along the vessel wall (while still leaving a patent lumen to enable blood flow through the blood vessel, through expandable structure 19, despite implantation of endovascular device 16), which can reduce the overall power needed to deliver efficacious electrical stimulation therapy to a target tissue site, and help secure electrodes 17 in place in the blood vessel for chronic therapy delivery.

Medical device 14 can be an external medical device or an implantable medical device that includes electrical stimulation circuitry configured to generate and deliver electrical stimulation therapy to patient 12 and/or sensing circuitry configured to sense a patient parameter (e.g., a physiological signal) via one or more electrodes 17 of endovascular device 16. In the example shown in FIG. 1, endovascular device 16 is directly or indirectly mechanically and electrically coupled to medical device 14 via a header 11 of medical device 14, which defines a plurality of electrical contacts in one or more feedthrough portions for electrically coupling electrodes 17 to electrical stimulation generation circuitry and/or sensing circuitry within medical device 14. In some examples, header 11 includes multiple feedthrough portions, which may be respectively configured for receiving one of multiple portions of endovascular device 16. Header 11 may also be referred to as a connector block or connector of medical device 14. Endovascular device 16 may be coupled to header 11 with the aid of a lead extension. However, in some examples, a lead extension is not used between header 11 and endovascular device 16, and endovascular device is directly mechanically and/or electrically connected to medical device 14 via header 11.

In some examples, medical device 14 is configured to be implanted in patient 12 in any suitable location, such as a location in a pectoral region. In other examples, medical device 14 is configured to be external to patient 12. Endovascular device 16 may be, for example, implanted within a vein (e.g., jugular vein 13) and one or more proximal wires/leads can remain within the venous system until they exit the venous system, such as through the subclavian vein in the chest or the internal jugular vein in the neck for implant in the pectoral region. In yet other examples, some or all of medical device 14 is configured to be implanted in the vasculature, e.g., as part of endovascular device 16.

As shown in FIG. 1, system 10 may also include a programmer 20, which may be a handheld device, portable computer, or workstation that provides a user interface to a user, for example a clinician or other user, such as a patient. The user may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include an electrode combination, a power, and an amplitude, which may be a current or voltage amplitude, and, if medical device 14 delivers electrical pulses, a pulse width, and a pulse rate for stimulation signals to be delivered to the patient. Other example therapy parameters include a slew rate, duty cycle, and phase of the electrical stimulation signal.

An electrode combination may include a selected subset of one or more electrodes 17 located on one or more implantable endovascular devices 16 coupled to medical device 14. The electrode combination may also refer to the polarities of the electrodes in the selected subset. By selecting particular electrode combinations, a user may target particular tissue sites (e.g., anatomic structures) within patient 12. In addition, by selecting values for slew rate, duty cycle, phase amplitude, pulse width, and/or pulse rate, the user can attempt to generate an efficacious therapy for patient 12 that is delivered via the selected electrode subset.

Whether programmer 20 is configured for clinician or patient use, programmer 20 may be configured to communicate with medical device 14 or any other computing device via wireless or a wired communication. Programmer 20, for example, may communicate via wireless communication with medical device 14 using radio frequency (RF) telemetry techniques. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

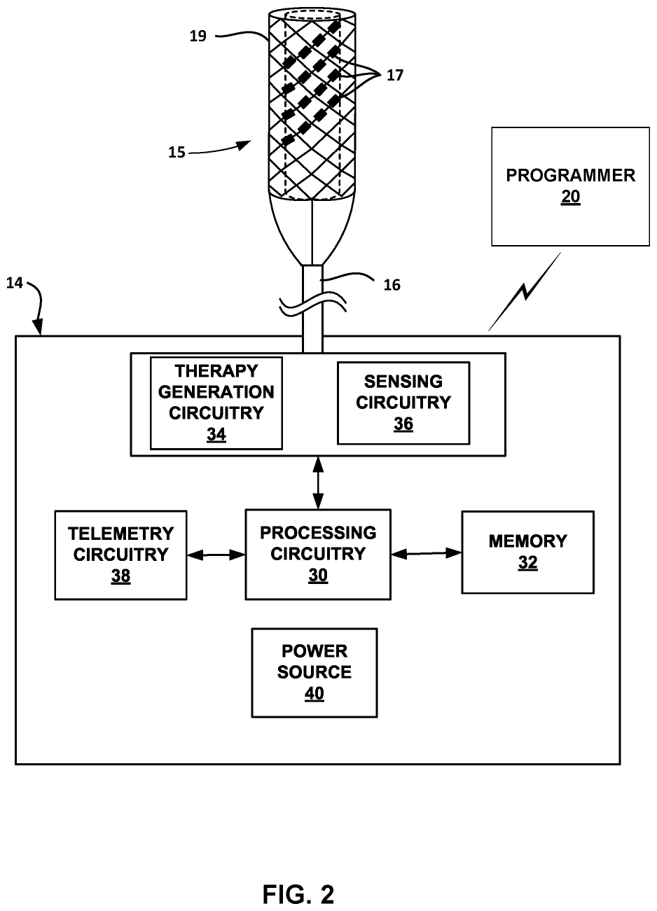

In some examples, in addition to or instead of delivering electrical stimulation to a target location (e.g., vagus nerve 21), medical device 14 or another device senses one or more patient parameters, such as bioelectrical signals, either using electrodes 17 or other types of sensors that are carried by endovascular device 16. Bioelectric signals can be sensed, and indications of sensed signals can be used by clinicians to make clinically relevant decision. In other examples, sense bioelectric signals are used as part of continuous feedback system in which medical device 14 adjusts one or more therapy parameter values based on sensed bioelectrical signals. Example bioelectric signals are described in further detail below with reference to FIG. 2.

In some examples, medical device 14 is configured to generate and deliver a suitable electrical stimulation signal, which can be a continuous time signal (e.g., a sinusoidal waveform or the like) or a plurality of pulses. In some examples, the electrical stimulation waveform generated by medical device 14 and delivered by one or more of electrodes 17 is a charge balanced, biphasic waveform. In some examples, such an electrical stimulation waveform consists of periodic pulses or otherwise include periodic pulses, or can include a continuous time waveform.

As noted above, in some examples, one or more electrodes 17 are positioned on expandable structure 19. In some examples, one or more sensors that are different from electrodes 17 are positioned on the same expandable structure (e.g., expandable structure 19) as one or more electrodes 17 or on a different expandable structure (e.g., a structure similar to or different from expandable structure 19) of endovascular device 16. Expandable structure 19 can have any suitable configuration that enables endovascular device 16 to assume a relatively low-profile configuration (also referred to herein as a “delivery” or “compressed” configuration in some examples) to facilitate delivery through vasculature to a target tissue site and expand radially outwards (relative to a central longitudinal axis of endovascular device 16) to position the one or more electrodes 17 closer to target tissue.

In some examples, expandable structure 19 is configured to expand radially outwards with sufficient force and to a cross-sectional dimension (e.g., a diameter) sufficient to position the one or more electrodes 17 in apposition with a blood vessel wall. Positioning one or more electrodes 17 in apposition with a blood vessel wall may help promote tissue ingrowth around electrodes 17, which can reduce the impedance and the overall power needed to deliver efficacious electrical stimulation therapy to a target tissue site, and help secure electrodes 17 in place in the blood vessel for chronic (e.g., on the order of months or even years) therapy delivery. Fixing endovascular device 16 in place within the blood vessel via the tissue ingrowth or, in some examples, using another fixation structures/anchoring mechanisms, such as tines, coils barbs, or the like, can also help reduce the possibility of thrombosis.

Expandable structure 19 can be configured to expand radially outwards using any suitable technique and configuration. In some examples, expandable structure 19 includes a shape memory (e.g., nitinol) material that enables the expandable structure to assume a predetermined shape in the absence of a force (e.g., a compressive or tensile force) holding expandable structure 19 in a relatively low-profile delivery configuration. For example, expandable structure 19 can be configured to expand radially outwards upon deployment from an outer sheath (e.g., an outer catheter), or upon the proximal withdrawal of a straightening element (e.g., a guidewire or a mandrel) positioned in an inner lumen of the endovascular device 16. In some examples, expandable structure 19 is configured to expand radially outwards in response to proximal withdrawal of a pull member attached to a distal portion of the endovascular device 16, in response to a distal movement of an elongated control member attached to the expandable structure, or with the aid of a balloon or the like.

Expandable structure 19 can have any suitable configuration in its deployed (e.g., expanded) configuration. In some examples herein, expandable structure 19 includes a plurality of interconnected struts to form a structure configured to expand radially outward (e.g., from a central longitudinal axis of expandable structure 19). For example, expandable structure 19 can include a tubular member, a basket, include one or more splines or arms configured to expand radially outwards, define one or more loops, define a helical or spiral element, or the like or combinations thereof, when in the deployed configuration. One or more expandable structures 19 may be disposed at various positions along endovascular device 16 (e.g., at one or more longitudinal positions along endovascular device 16). Expandable structure 19 can be formed from a plurality of structural elements (e.g., braided or coupled together) or can be a unitary structure (e.g., a laser cut nitinol tube).

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 (as well as electrodes 17, medical device 14, processing circuitry, etc.) may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration than a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more endovascular devices may be used for multiple trial periods (e.g., successive trial periods) for determining an efficacy of one or more stimulation parameters and/or one or more sensing parameters.

Endovascular device 16 may have any suitable configuration for delivering electrical stimulation to a target tissue site in patient 12 or sensing a patient parameter from an endovascular location (e.g., jugular vein 13). In some examples, endovascular device includes a first subset of electrodes of electrodes 17 configured for delivering electrical stimulation therapy and a second subset of electrodes of electrodes 17 configured to for sensing one or more patient parameters. In some examples, some or all electrodes of electrodes 17 are configured for both electrical stimulation therapy and for sensing one or more patient parameters. Endovascular device 16 can include any suitable number of electrodes 17 and/or combination of different kinds of electrodes. In some examples, electrodes 17 include electrodes formed via one or more manufacturing processes. For example, electrodes 17 can include a first electrode type (e.g., one or more stamped electrodes), a second electrode type (e.g., one or more machined electrodes), or any suitable combination thereof.

FIG. 2 is a functional block diagram illustrating components of an example medical device 14, which is configured to generate and deliver electrical stimulation therapy to patient 12 and, in some examples, sense one or more patient parameters, such as bioelectrical signals or other physiological parameter of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate and deliver electrical stimulation signals to target tissue (e.g., vagus nerve 21) in patient 12. Processing circuitry 30 is configured to control therapy generation circuitry 34 to generate and deliver electrical stimulation therapy via electrodes 17 of endovascular device 16. The therapy parameter values may be selected based on the patient condition being addressed, as well as the target tissue site in patient 12 for the electrical stimulation therapy. The electrical stimulation therapy can be provided via stimulation signals of any suitable form, such of stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from electrodes 17 configured to deliver electrical stimulation therapy. In some examples, processing circuitry 30 stores the sensed physiological parameters in memory 32 or transmits the sensed parameters to another device via telemetry circuitry 38. In addition, in some examples, processing circuitry 30 can use the sensed physiological signals to control therapy delivery by therapy generation circuitry 34, e.g., the timing of the therapy delivery or one or more characteristics (e.g., parameters values) of the electrical simulation signal generated by therapy generation circuitry 34.

In some examples, sensing circuitry 36 is configured to sense a bioelectrical signal, which otherwise may be referred to as a patient parameter, via one or more electrodes 17 (e.g., all or a subset of electrodes 17). Thus, electrodes 17 can be configured to receive or transmit energy (e.g., current). In some examples, such as those in which electrodes 17 are placed proximate vagus nerve 21 (FIG. 1), example bioelectric signals include muscle activation signals (e.g., laryngeal muscle activation), electrocardiogram (ECG), intracardiac electrogram (EGM), electromyogram (EMG). In other examples, such as those in which electrodes 17 are placed in or otherwise proximate brain 18, example bioelectrical signals include brain signals such as an EEG signal, an electrocorticogram (ECoG) signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry 34, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals are processed by an analog-to-digital converter of processing circuitry 30 or other component to convert the conditioned analog signals into digital signals. In some examples, processing circuitry 30 operates on the analog or digital form of the signals to separate out different components of the signals. In some examples, sensing circuitry 36 and/or processing circuitry 30 performs any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

In some examples, processing circuitry 30, alone or in combination with therapy generation circuitry 34 and/or sensing circuitry 36, is configured to operate medical device 14 (including electrodes 17, endovascular device 16, etc.) in a trial mode for a trial period to determine an efficacy of electrical stimulation or sensing. As described above, a trial mode can include a trial period of stimulation and/or sensing to determine, evaluate, or confirm an efficacy of stimulation and/or sensing. In some examples, processing circuitry 30, alone or in combination with therapy generation circuitry 34 and/or sensing circuitry 36, is configured to deliver electrical stimulation therapy and/or sense a patient parameter during the trial period. In some examples, processing circuitry 30 is configured to determine, evaluate, or confirm an efficacy of stimulation and/or sensing. For example, processing circuitry 30 may determine one or more therapy parameters for chronic stimulation and/or sensing based on the trial period.

Although shown as part of medical device 14 in FIG. 2, in other examples, sensing circuitry 36 is part of a device separate from medical device 14. For example, sensing circuitry 36 can be part of an implantable sensing device implanted in patient 12.

Processing circuitry 30, as well as other processors, processing circuitry, controllers, control circuitry, and the like, described herein, may include any combination of integrated circuitry, discrete logic circuity, analog circuitry, such as one or more microprocessors, digital signal processors (DSPs), application specific integrated circuits (ASICs), or field-programmable gate arrays (FPGAs). In some examples, processing circuitry 30 includes multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20 (FIG. 1), may accomplish communication by any suitable communication techniques, such as RF communication techniques. In addition, telemetry circuitry 38 may communicate with external medical device programmer 20 via proximal inductive interaction of medical device 14 with programmer 20. Accordingly, telemetry circuitry 38 may send information to external programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

In some examples, endovascular device 16 is configured to be a standalone electrical stimulation device and can include one or more elements of medical device 14 shown in FIG. 2.

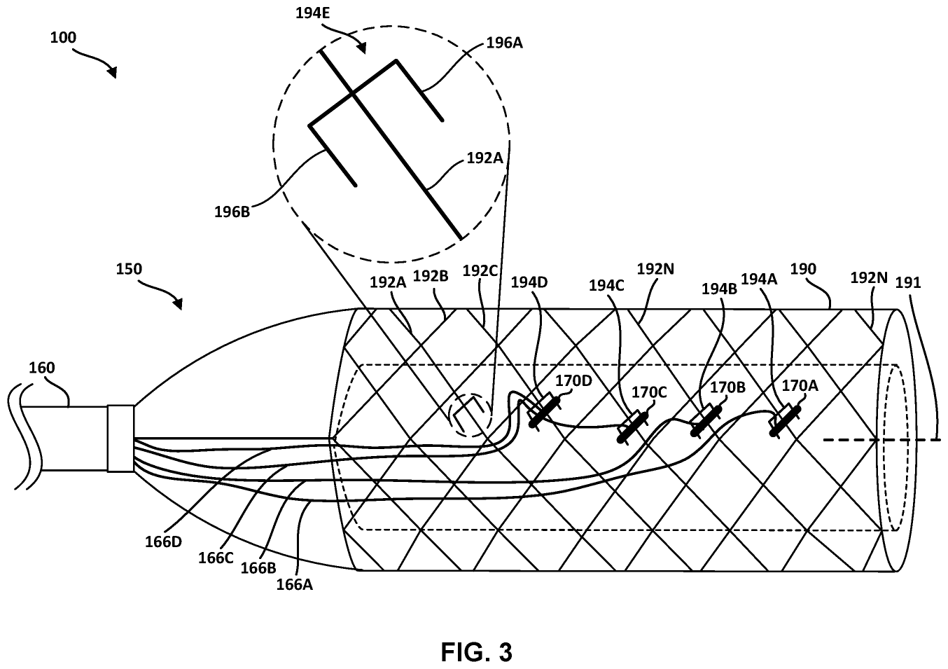

FIG. 3 illustrates an example endovascular therapy system 100, which is an example of therapy system 10 of FIG. 1. Endovascular therapy system 100 includes a medical lead 160 and an expandable structure 190 at a distal portion 150 of medical lead 160. As shown, endovascular therapy system 100 includes electrode 170A, electrode 170B, electrode 170C, and electrode 170D, collectively referred to herein as electrodes 170. Medical lead 160, expandable structure 190, distal portion 150, and electrodes 170 may be examples of endovascular device 16, expandable structure 19, distal portion 15, and electrodes 17 as shown and described in connection with FIG. 1, respectively.

In some examples, expandable structure 190 is configured to position and/or orient electrodes 170 within vasculature of a patient. In some examples, electrodes 170 are carried by and/or disposed on by expandable structure 190, and expandable structure 190 is configured to transform from a relatively low-profile delivery configuration to a deployed configuration in a blood vessel of a patient (e.g., within jugular vein 13 of patient 12 as discussed in relation to FIG. 1). Expandable structure 190 is an example of expandable structure 19 as discussed in connection with FIG. 1, and can include any suitable shape and materials. In some examples, expandable structure 190 includes one or more of a self-expanding stent, a self-expanding coil, or another suitable expandable structure that includes one or more struts as described herein.

Expandable structure 190 can have any suitable configuration for positioning electrodes 170 for delivering stimulation therapy and/or sensing one or more patient parameters of patient 12 from an endovascular location. In some examples, as shown in FIG. 3, expandable structure 190 includes a body portion includes a plurality of interconnected struts 192 (shown individually as strut 192A, strut 192B, strut 192C . . . strut 192N, but collectively referred to herein as struts 192). In some examples, struts 192 form a tubular (e.g., stent-like) structure. Expandable structure 190 defines a central longitudinal axis 191. In some examples, expandable structure 190 is configured to expand (e.g., self-expand and/or via an expansion mechanism such as a balloon) radially outward from central longitudinal axis 191 to a deployed configuration, such as to position electrodes 170 into apposition with a blood vessel wall (e.g., for delivering electrical stimulation therapy to tissue of patient 12 surrounding the blood vessel and/or sensing a patient parameter from a location within the blood vessel).

In the example of FIG. 3, expandable structure 190 includes structural features to facilitate mechanical coupling of electrodes 170 to expandable structure 190 as well as orient electrodes 170 with respect to expandable structure 190. As shown in FIG. 3, expandable structure 190 includes electrode attachment element 194A, electrode attachment element 194B, electrode attachment element 194C, electrode attachment element 194D, and electrode attachment element 194E, collectively referred to herein as electrode attachment elements 194. Each electrode attachment element 194A-194E may be configured to couple to one or more electrodes of electrodes 170 and orient electrodes 170 with respect to expandable structure 190. In some examples, each electrode attachment element 194A-194E is configured to minimize rotation (e.g., reduce or even eliminate rotation) of a given electrode of electrodes 170 around one or more of struts 192 (e.g., by fixing an orientation of a given electrode 170 with respect to one or more of struts 192).

In some examples, expandable structure 190 includes a suitable number of electrode attachment elements 194 configured to mechanically couple electrodes 170 to expandable structure 190. As shown in the example of FIG. 3, expandable structure 190 includes five electrode attachment elements 194. However, expandable structure 190 can include any suitable number of electrode attachment elements 194 (e.g., one, two, three, four, five, six, seven, eight, nine, ten, twelve, fifteen, twenty, thirty, etc.). In some examples, expandable structure 190 includes more electrode attachment elements than electrodes 170 that are eventually affixed to expandable structure 190, such that expandable structure 190 can be pre-fabricated, and a suitable number, configuration, and pattern of electrodes 170 can subsequently be attached to expandable structure 190 depending on the end use of endovascular therapy system 100. For example, expandable structure 190 can include electrode attachment elements 194 at multiple circumferential positions around expandable structure 190 and/or multiple longitudinal positions along expandable structure 190 (e.g., spaced apart along central longitudinal axis 191). In some of these examples, not all the electrode attachment elements 194 will be used to couple electrodes 170 to expandable structure 190, but some electrode attachment elements 194 will remain unattached to electrodes 170 after all of the desired number of electrodes 170 are coupled to expandable structure 190.

Electrode attachment elements 194 can each include suitable configurations for mechanically receiving and/or otherwise mechanically coupling to one or more electrodes of electrodes 170. Similarly, electrodes 170 can includes suitable configuration for mechanically receiving and/or otherwise mechanically coupling to electrode attachment elements 194. In some examples, as shown in the example of FIG. 3, each electrode attachment element 194A-194D includes at least a first projection 196A and a second projection 196B (collectively referred to herein as projections 196). In some examples, each of first projection 196A and/or second projection 196B are configured to couple to mating portions of one or more of electrodes 170. In some examples, projections 196 are formed (e.g., integrally formed) as part of a unitary structure of expandable structure 190 along with interconnected struts 192. In some examples, expandable structure 190, including interconnected struts 192 and electrode attachment elements 194, is a single, continuous structure. For example, expandable structure 190, including interconnected struts 192 and electrode attachment elements 194, can be laser-cut from a single piece of material (e.g., nitinol, or another metallic material).

In other examples, electrode attachment elements 194 (e.g., which each can include first projection 196A and second projection 196B) are formed separately from expandable structure 190, and subsequently attached to expandable structure 190. For example, electrode attachment elements 194 (e.g., which each can include first projection 196A and second projection 196B) are separately formed and attached to respective struts of struts 192 (e.g., electrode attachment element 194E including first projection 196A and second projection 196B is formed separately from expandable structure 190 and subsequently attached to strut 192A of expandable structure 190). When formed separately from expandable structure 190, electrode attachment elements 194 can include a different material as expandable structure 190 (e.g., of struts 192), and/or different dimensions (e.g., cross-sectional dimensions) as struts 192.

While the example of FIG. 3 shows electrode attachment elements 194 having two projections (e.g., first projection 196A and second projection 196B), electrode attachment elements 194 can include more than two projections 196, such as for facilitating mechanical connection of electrodes 170 to expandable structure 190 as described in this disclosure. In some examples, electrode attachment elements 194 include a corresponding number of projections 196 as respective mating structural features (e.g., holes and/or other structural features) on electrodes 170.

Projections 196 of each electrode attachment elements 194A-194D can include any suitable configuration for mechanically coupling electrodes 170 to expandable structure 190. In the example of FIG. 3, electrode attachment elements 194A-194D are longitudinally spaced apart long expandable structure 190 (e.g., along central longitudinal axis 191) such that respective projections 196 of each of electrode attachment elements 194A-194D branch off of different struts of struts 192A-192N. That is, each set of projections 196 (including first projection 196A and second projection 196B) branch off of different struts of struts 192A-192N. As shown in the exploded view of electrode attachment element 194E, first projection 196A and second projection 196B branch off of strut 192A of expandable structure 190 in opposite directions from strut 192A, such that first projection 196A and second projection 196B are on an opposite sides of strut 192A. Said another way, first projection 196A extends away from a first side of strut 192A and second projection 196B extends away from a second side of strut 192A opposite the first side. In some examples, first projection 196A and second projection 196B are symmetrical (e.g., reflectionally symmetric) about strut 192A. In other examples, first projection 196A and second projection 196B are asymmetrically positioned relative to strut 192A.

Projections 196 can have other suitable configurations different than the configuration illustrated in FIG. 3. For example, in some examples, first projection 196A and second projection 196B branch off of strut 192A such that both first projection 196A and second projection 196B are the same side of strut 192A. Further, branches forming a single electrode attachment element 194 may branch off of different struts of interconnected struts 192 (e.g., strut 192A and strut 192B), such that one or more of electrodes 170 are held between struts of struts 192. In some examples, first projection 196A and second projection 196B are positioned at a junction between intersecting struts of struts 192 (e.g., at a junction between strut 192A and strut 192B). In some examples, each of first projection 196A and second projection 196B reside at approximately the same radial distance away from central longitudinal axis 191. In some examples, first projection 196A is closer to central longitudinal axis 191 as compared to second projection 196B, or vice versa.

In some examples, projections 196 of each electrode attachment element 194 include a suitable shape and size for coupling electrodes 170 to expandable structure 190. In some examples, each of first projection 196A and second projection 196B can each include a first portion that is parallel or substantially parallel (e.g., to the extent permitted by manufacturing tolerances) to strut 192A and a second portion that is perpendicular or substantially perpendicular (e.g., to the extent permitted by manufacturing tolerances) to strut 192A. For example, as shown in the example of FIG. 3, each of first projection 196A and second projection 196B forms an L-shape (e.g., a right-angle L-shape). In some examples, each of first projection 196A and second projection 196B forms a curve (e.g., such that each of first projection 196A and second projection 196B forms a continuous curve). In some examples, each of first projection 196A and second projection 196B forms a curved L-shape. A maximum distance between each of first projection 196A and second projection 196B can correspond to respective mating structural features (e.g., holes or apertures) on electrodes 170.

As described later in more detail, in some examples, some or all of electrodes 170 are configured to receive projections 196 of respective electrode attachment elements 194. For example, although an electrode is not shown in conjunction with electrode attachment element 194E, an electrode of electrodes 170 can be configured to receive each of first projection 196A and second projection 196B via structural features (e.g., holes, apertures, etc.) defined by the respective electrode 170. In some examples, once electrodes 170 have received respective projections 196 of electrode attachment elements 194, electrodes 170 are fixedly coupled to the respective projections 196 via one or more of welding, adhesive, crimping, interference fit and/or another suitable method. In this way, each of electrodes 170 are configured to couple to expandable structure 190 via respective electrode attachment elements 194.

In some examples, such as the example of FIG. 3, electrodes 170 are configured to receive projections 196 such that projections 196 extend entirely though electrodes 170. As shown in FIG. 3, at least a portion of each of first projection 196A and second projection 196B extends away from each of electrodes 170 (e.g., on opposite sides of each of electrodes 170). As described more in relation to later examples, each of electrodes 170 can define holes extending entire through an electrode body of each of electrodes 170 for receiving projections 196. In other examples, each of electrodes 170 define holes extending only partially through the body of each of electrodes 170 (e.g., blind holes). In some examples, some holes of each of electrodes 170 are blind holes, while other holes extend entirely through electrodes 170 (e.g., holes for receiving projections 196 and/or conductor wires 166).

Endovascular therapy system 100 including electrodes 170 coupled to expandable structure 190 may be configured to have a relatively low-profile configuration to facilitate delivery and/or placement into relatively narrow and/or tortuous vessels. Once proximate a target location, expandable structure 190 can be configured to transform to the deployed configuration. In some examples, when expandable structure 190 is in a deployed configuration, electrodes 170 are flush or nearly flush with an outer surface of expandable structure 190. In some examples, electrode attachment elements 194 are configured to position electrodes 170 such that at least one surface of each of electrodes 170 is flush or nearly flush with the outer surface of expandable structure 190.

In the example of FIG. 3, system 100 includes conductor wires 166 (shown individually as conductor wire 166A, conductor wire 166B, conductor wire 166C, and conductor wire 166C, but collectively referred to as conductor wires 166) configured to electrically connect electrodes 170 to a medical device (e.g., medical device 14 in the example of FIG. 1). In some examples, each electrode 170A-170D is configured to receive one or more conductor wires of conductor wires 166A-166D. For example, as described in more detail in relation to later examples, each electrodes 170A-170D can define one or more conductor holes configured to receive one or more conductor wires 166A-166D for electrically coupling respective electrodes to a medical device (e.g., medical device 14 in the example of FIG. 1). The holes in each of electrodes 170 for receiving and/or electrically connecting to one or more conductor wires 166 can extend partially or entirely though each of electrodes 170.

In some examples, one or more of electrodes 170A-170D can define a conductor hole configured as a pass-through hole to facilitate electrical connection of the conductor wire to a second, different electrode of one or more of electrodes 170A-170D. For example, as shown in FIG. 3, electrode 170D (which may be a proximal-most electrode) can define a conductor hole configured as a pass-through hole, such that a conductor wire (e.g., as shown, conductor wire 166C) that also connects to a more distal electrode (e.g., electrode 170C) passes through the conductor hole of electrode 170D. In this way, in examples where at least some of electrodes 170 are not connected to a common conductor wire of conductor wires 166 (e.g., as shown in FIG. 3), some or all of electrodes 170 may still include a conductor pass-through hole, such as for managing and/or routing conductor wires to other electrodes of electrodes 170. In this way, at least one of the electrodes 170 can be configured to help hold the conductor wires relatively close to expandable structure 190 and out of the way of blood flowing through a lumen of expandable structure 190, which may help minimize thrombus formation. Additionally or alternatively, multiple of electrodes 170 can be electrically connected to a common conductor wire of conductor wires 166, such that more than one of electrodes 170 can be controlled together (e.g., “shorted” together such that a medical device, such as medical device 14 in the example of FIG. 1, can control electrodes 170 simultaneously).

Medical lead 160 can have any suitable configuration, and may be configured according to the description of endovascular device 16 in the example of FIG. 1. In some examples, medical lead 160 includes a lead body that is configured to be at least partially implanted in vasculature of patient 12. In some examples, lead 160 includes an electrically insulative material covering at least some portions of lead 160. The insulative material can be configured to electrically insulate portions of conductor wires 166 that run along the length of lead 160. Expandable structure 190 can be carried by or otherwise affixed to medical lead 160 using any suitable technique. In some examples, expandable structure is welded to a portion of medical lead 160. In some examples a band (e.g., a markerband) of material or ring is configured to couple expandable structure 190 to lead 160. In general, expandable structure 190 can be positioned at a distal portion of medical lead 160. In some examples, expandable structure 190 is positioned at a distal end of lead 160.

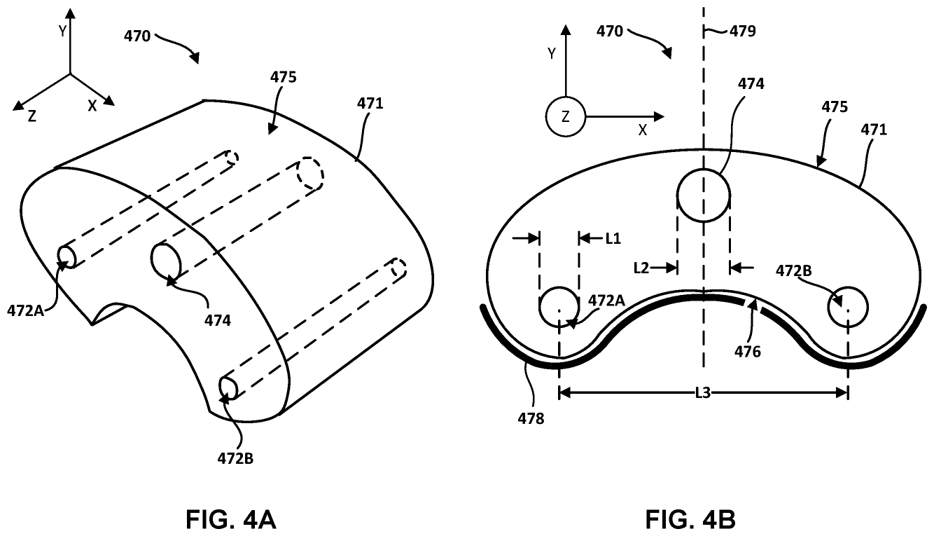

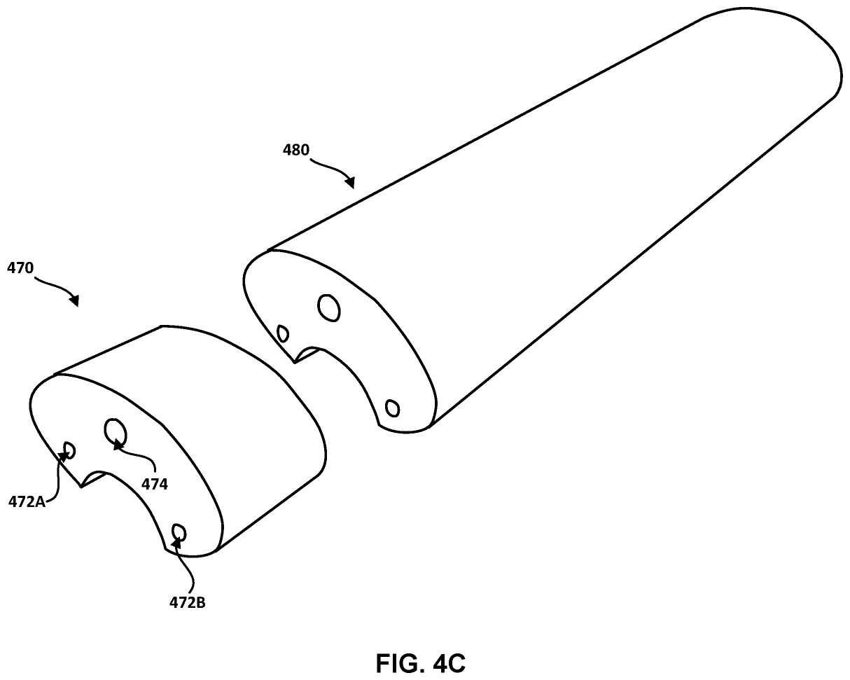

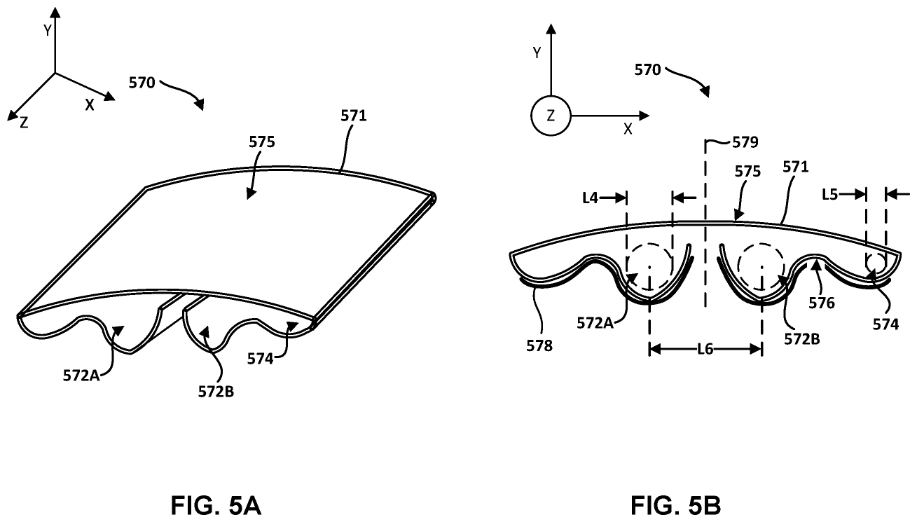

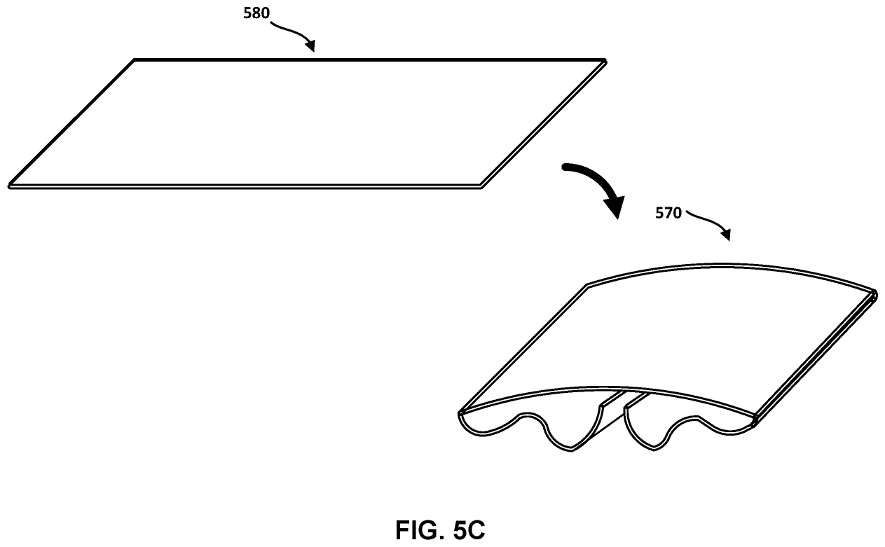

Electrodes 170 can be fabricated using any suitable method, and some of all of electrodes 170 can be formed according to the method described herein. As described in connection with FIG. 4A, FIG. 4B, and FIG. 4C, some or all of electrodes 170 can include machined electrodes. As described in connection with FIG. 5A, FIG. 5B, and FIG. 5C, some or all of electrodes 170 can include stamped electrodes. In some examples, some or all of electrodes 170 can include a combination of machined electrodes and stamped electrodes. In some examples, certain types of electrodes may be used for different purposes (e.g., some for delivering electrical stimulation therapy, some for sensing a patient parameter, and some for both delivering electrical stimulation therapy and for sensing a patient parameter).

Although FIG. 3 is described with respect to electrodes 170 that are configured to deliver electrical stimulation therapy and/or sense electrical signals, endovascular therapy system 100 can additionally or alternatively include other types of therapy delivery elements and/or sensors. In some examples, endovascular therapy system 100 includes one or more ultrasound transducers, chemical delivery elements (e.g., fluid delivery elements) which can be configured to be attached to expandable structure 190 using a similar method of attachment as electrodes 170. In some examples, endovascular therapy system 100 additionally or alternatively includes one or more temperature sensors, pressure sensors, optical sensors, impedance sensors, chemical sensors, and/or other suitable types of sensors, which can be configured to be attached to expandable structure 190 using a similar method of attachment as electrodes 170.

FIG. 4A-4C and FIG. 5A-5C illustrate machined electrode 470 and stamped electrode 570, respectively, which are examples of electrodes suitable for use as electrodes 17 and/or electrodes 170 described in connection with FIG. 1 and FIG. 3 respectively. In particular, FIG. 4A, FIG. 4B, and FIG. 4C illustrate an example machined electrode and FIG. 5A, FIG. 5B, and FIG. 5C illustrate an example stamped electrode. As described herein, the terms “machined” and “stamped” refer to manufacturing techniques for fabricating components, such as electrodes. In some examples, the manufacturing technique can impart structural characteristics (e.g., unique structural characteristics depending on the type of manufacturing techniques). However, the electrodes described in this disclosure may be fabricated according to other manufacturing and/or processing techniques to achieve the same configuration (e.g., form factor) of electrodes described in this disclosure.

FIG. 4A and FIG. 4B illustrate a perspective view and a side view, respectively, of a machined electrode 470, which may be an example of one of electrodes 17 from FIG. 1 and/or one of electrodes 170 from the example of FIG. 3. Machined electrode 470 includes an electrode body 471 that defines at least one electrically conductive surface and structural features configured to help attach machined electrode 470 to a portion of a medical lead and/or other structure (e.g., medical lead 160 and/or expandable structure 190, as shown and described with respect to FIG. 3). For illustrative purposes, some elements described in FIG. 3 will be referenced herein with respect to machined electrodes 470 (e.g., as corresponding mating structural features), however it should be understood that one or more of machined electrode 470 is only one example of electrodes 170 of FIG. 3, and that other types of electrodes can additionally or alternatively be used in conjunction with the elements described in connection with FIG. 3. For example, electrode attachment element 194E, including first projection 196A and second projection 196B, as well as strut 192A of interconnected struts 192 of FIG. 3 will be referenced to illustrate how machined electrode 470, shown in FIG. 4A and FIG. 4B, can be configured to attach to expandable structure 190 of FIG. 3.

In some examples, machined electrode 470 includes a suitable electrically conductive material configured for transmitting and/or receiving electrical signals. In some examples, machined electrode 470 includes one or more of titanium (Ti), tantalum (Ta), Tin (Sn), and/or a suitable combination thereof (e.g., TiTaSn and/or similar alloys). In some examples, machined electrode 470 includes one or more of platinum (Pt) and/or Iridium (Ir) and/or a suitable combination thereof (e.g., PtIr and/or similar alloys).

As shown in the example of FIG. 4A and FIG. 4B, a portion of machined electrode 470 defines a first surface 475, which may be an electrically conductive surface configured to transmit and/or receive electrical signals. First surface 475 can be configured to transmit and/or receive electrical signals, such as examples where machined electrode 470 is positioned on expandable structure 190 of endovascular therapy system 100 (e.g., where endovascular therapy system 100 is configured for endovascular stimulation therapy and/or sensing a patient parameters). When affixed to expandable structure 190, first surface 475, which may be a conductive surface, is configured to face radially outward from expandable structure 190, such as in a direction radially outward from central longitudinal axis 191 of expandable structure 190 in the example of FIG. 3. In the examples of FIG. 4A and FIG. 4B, first surface 475 faces in the positive “y” direction according to the orthogonal x-y-z axes shown in FIG. 4A and FIG. 4B.

In some examples, first surface 475 includes a coating or other surface treatment to facilitate transmitting and/or receiving electrical signals. In some examples, first surface 475 includes a coating comprising one or more of Titanium Nitride (TiN), Iridium Oxide (IrOx), another suitable material, or a combination thereof. In some examples, first surface 475 additionally or alternatively includes a surface treatment, such as laser texturing to facilitate transmitting and/or receiving electrical signals. The surface treatment (e.g., laser texturing or mechanical texturing) can help effectively increase the conductive surface area of first surface 475 for a given footprint of machined electrode 470.

As shown in the example of FIG. 4B, a portion of machined electrode 470 defines a second surface 476, which may opposite first surface 475 (e.g., on an opposite side of electrode body 471). In some examples, an electrically insulative material 478 is applied to at least a portion of second surface 476 (e.g., all or part of second surface 476), which may reduce or inhibit transmission of electrical signals to and/or from second surface 476 of machined electrode 470. When machined electrode 470 is affixed to expandable structure 190, second surface 476, which may include an insulative material, is configured to face radially inward from expandable structure 190, such as in a direction radially inward toward central longitudinal axis 191 of expandable structure 190. Electrically insulative material 478 may reduce or even prevent transmission of electrical signals to and/or from less desirable regions of tissue and/or untargeted regions of tissue (e.g., a radially inward portion of a blood vessel). In this way, machined electrode 470 can bias transmissions of electrical signals to and/or from tissue surrounding the blood vessel as compared to radially inward from the blood vessel wall. In some examples, insulative material 478 extends beyond second surface 476, such that insulative material 478 is applied to one or more sides of the electrode body 471 (sides of machined electrode 470 different than first surface 475 and/or second surface 476).

In some examples, machined electrode 470 is configured to bias transmissions of electrical signals to and/or from tissue surrounding the blood vessel (jugular vein 13) as compared to radially inward from the blood vessel wall. For example, in examples where machined electrode 470 includes first surface 475 (which may be an electrically conductive surface facing radially outward towards a blood vessel wall) and second surface 476 (which may include an electrically insulative material facing radially inward from a blood vessel wall), machined electrode 470 is configured to bias transmission of electrical signals to tissue surrounding the blood vessel in which expandable structure 190 is positioned. The biasing of transmissions of electrical signals to and/or from tissue surrounding the blood vessel can help facilitate directional electrical stimulation and/or sensing.

In some examples, at least a portion of second surface 476 is configured to interface with (e.g., contact) a strut (e.g., strut 192A) of an expandable structure (e.g., expandable structure 190). For example, when machined electrode 470 is attached to expandable structure 190, at least a portion of second surface 476 is in contact with (e.g., rests on) a strut (e.g., strut 192A) of expandable structure 190. In some examples, at least a portion of second surface 476 defines a shape (e.g., a concave shape) that conforms to strut 192A. In some examples, a radius of curvature of at least a portion of second surface 476 is equal to or approximately equal to (e.g., to account for manufacturing tolerances) a radius of curvature of strut 192A (e.g., a radius of curvature of a cross-section of a strut 192A). In some examples, a radius of curvature of at least a portion of second surface 476 is greater than a radius of curvature of strut 192A. Configuring second surface 476 to conform to a surface of strut 192A and/or expandable structure 190 can help enable machined electrode 470 to have a relatively low profile, which can help reduce the overall profile of expandable structure 190 and the space occupied by expandable structure 190 in a blood vessel. This may help reduce any adverse impacts on blood flow through the blood vessel.

Machined electrode 470 can include a suitable shape, size, and/or configuration for delivering electrical stimulation therapy and/or sensing bioelectric signals from an endovascular location. In some examples, machined electrode 470 (e.g., machined electrode body 471) defines a non-cylindrical shape. In some examples, the non-cylindrical shape of machined electrode 470 facilitates directional electrical stimulation therapy and/or directional sensing, such as in a direction radially outward from expandable structure 190 and/or radially outward relative to a blood vessel (e.g., jugular vein 13) and towards a blood vessel wall. For example, the non-cylindrical shape (e.g., as compared to a cylindrical electrode shape) can facilitate easier application of an insulative material to one side of machined electrode 470 (e.g., second surface 476) while leaving another side (e.g., first surface 475) for transmitting and/or receiving electrical signals. The non-cylindrical shape of machined electrode 470 can facilitate easier processing during manufacturing and/or assembly, such as by allowing for fixturing for application of an insulative material to only one side of machined electrode 470 (e.g., second surface 476) and/or for processing another side of machined electrode 470 (e.g., first surface 475) to enhance transmission of electrical signals to and/or from machined electrode 470.

In some examples, a major surface (e.g., a major conductive surface, such as first surface 475) of machined electrode 470 is configured to transmit and/or receive electrical signals toward a vessel wall. In some examples, first surface 475 is configured to be placed proximate (e.g., in apposition with) a vessel wall, such as to maintain contact against a vessel wall for electrical stimulation therapy and/or sensing. In some examples, at least a portion of first surface 475 is convex, and includes a radius of curvature similar to expandable structure 190 in a deployed configuration and/or similar to the blood vessel in which expandable structure 190 is configured to be placed. However, first surface 475 can define other shapes, including (but not limited to) flat, concave, irregular, and/or another shape.

In some examples, first surface 475 defines a suitable surface area for delivering stimulation therapy and/or sensing bioelectrical signals from an endovascular location, such as, but not limited to, a surface area of about 0.5 square millimeters (mm²) to about 10 mm², such as about 1.7 mm² to about 6 mm², or overlapping ranges thereof. However, in other examples, first surface 475 defines a surface area larger or smaller than these ranges, and a suitable surface area may depend on the electrode material and/or coating applied to machined electrode 470. For example, in some examples, first surface 475 defines a surface area of about 0.5 mm² to about 10 mm², or within a subrange thereof. In some examples, the surface area defined by one or more of machined electrode 470 facilitates delivery of electrical stimulation therapy and/or sensing from an endovascular location while also enabling the overall form-factor of machined electrode 470 to be relatively low-profile. For example, expandable structure 190 with one or more of machined electrode 470 can be collapsed down to a relatively small form factor in the delivery configuration of endovascular therapy system 100 (e.g., such that expandable structure 190 and one or more of machined electrode 470 can fit into a suitable delivery catheter).

In some examples, machined electrode 470 is configured to receive structural features of a medical lead and/or other structures (e.g., features of medical lead 160 and/or expandable structure 190, as shown and described with respect to FIG. 3), such as for mechanically coupling one or more of machined electrode 470 to medical lead 160 and/or another structure (e.g., expandable structure 190). The mechanical coupling of one or more of machined electrode 470 to expandable structure 190 according to the techniques of this disclosure can help orient machined electrode 470 with respect to the vasculatures such that first surface 475, which may be a conductive surface, faces radially outward and towards a vessel wall when expandable structure 190 with machined electrode 470 are placed and deployed within the vasculature.

In some examples, machined electrode 470 includes structural features configured to receive portions of an expandable structure (e.g., expandable structure 190, as shown and described with respect to FIG. 3). In the example of FIG. 4A and FIG. 4B, machined electrode 470 (e.g., electrode body 471) defines a first fixation hole 472A and a second fixation hole 472B (collectively referred to herein as fixation holes 472), which may be configured to receive respective portions of electrode attachment elements 194, as shown and described with respect to FIG. 3. For example, in some examples, first fixation hole 472A is configured to receive first projection 196A of electrode attachment element 194E, and second fixation hole 472B is be configured to receive second projection 196B of electrode attachment element 194E. In this way, machined electrode 470 is fixed via at least two connection points to expandable structure 190, which may minimize, or even prevent, rotation of machined electrode 470 with respect to (e.g., about) strut 192A.

Fixation holes 472 may be sized, oriented, and otherwise configured for receiving mating portions of expandable structure 190 (e.g., first projection 196A and second projection 196B of electrode attachment element 194E). In some examples, each of first fixation hole 472A and second fixation hole 472B defines an a maximum dimension L1. Maximum dimension L1 may be a diameter in examples where first fixation hole 472A and/or second fixation hole 472B defines a circular or approximately circular shape (e.g., a circular or approximately circular cross-section in a plane defined by the x and y axes in the example of FIG. 4B). In some examples, first fixation hole 472A is spaced apart from second fixation hole 472B by a distance L3. In some examples, the distance L3 extends between center points of each of first fixation hole 472A and second fixation hole 472B. Distance L3 between first fixation hole 472A and second fixation hole 472B may correspond to respective mating structural features of electrode attachment element 194E. In some examples, distance L3 corresponds to (e.g., is equal to or nearly equal to) a distance between first projection 196A and second projection 196B of electrode attachment element 194E.

In some examples, as shown in the example of FIG. 4A and FIG. 4B, each of first fixation hole 472A and second fixation hole 472B extend entire through machined electrode 470 (e.g., extend entirely through electrode body 471 in a direction along the z-axis according to the orthogonal x-y-z axes shown in FIG. 4A and FIG. 4B). In other examples, one or more of first fixation hole 472A and/or second fixation hole 472B extend only partially through machined electrode 470 (e.g., only partially through electrode body 471 in a direction along the z-axis), such that first fixation hole 472A and/or second fixation hole 472B are blind holes.

In some examples, machined electrode 470 is configured to electrically connect to one or more conductor wires, such as to facilitate electrical communication of machined electrode 470 with a medical device (e.g., medical device 14, as shown and described in connection with FIG. 1). In some examples, a portion of machined electrode 470 (e.g., electrode body 471) defines a conductor hole 474, where conductor hole 474 is configured to receive a conductor wire (e.g., one or more of conductor wires 166 as described with respect to FIG. 3) for electrically coupling machined electrode 470 to a medical device. In some examples, conductor hole 474 extends entirely through machined electrode 470 (e.g., extending entirely through electrode body 471 in a direction along the z-axis according to the orthogonal x-y-z axes shown in FIG. 4A and FIG. 4B). In other examples, conductor hole 474 extends only partially through machined electrode 470 (e.g., only partially through electrode body 471 in a direction along the z-axis), such that conductor hole 474 is a blind hole.

In some examples, conductor hole 474 is sized to accommodate one conductor wire (e.g., of conductor wires 166). In some examples, conductor hole 474 defines a maximum dimension L2. In some examples, maximum dimension L2 is slightly larger than a width (e.g., a maximum cross-sectional dimension) of one conductor wire (e.g., of conductor wires 166). In examples where conductor hole 474 defines a circular or approximately circular shape (e.g., a circular or approximately circular cross-section in a plane defined by the x and y axes in the example of FIG. 4B), maximum dimension L2 is a diameter.

In some examples, conductor hole 474 is configured as a pass-through hole for one or more conductor wires (e.g., conductor wires 166, as described with respect to FIG. 3). Conductor hole 474 being configured as a pass-through hole can facilitate electrical connection of the conductor wire (e.g., one or more of conductor wires 166) to a second, different electrode. For example, one or more of conductor wires 166 may be fed through conductor hole 474 and electrically connected to a different (e.g., more distal) electrode. In this way, multiple of machined electrodes 470 can be electrically connected to a common conductor wire of conductor wires 166 (e.g., “shorted together”), such that more than one of machined electrode 470 can be controlled together (e.g., by a medical device, such as medical device 14 in the example of FIG. 1). In some examples, machined electrode 470 includes more than one of conductor hole 474, such as in examples where one or more holes are configured for electrically connecting a conductor wire to machined electrode 470 and one or more additional holes are configured as pass-through holes for routing conductor wires to at least a second, different electrode (e.g., in examples where electrodes are not “shorted” together and are controlled separately by medical device 14).

In some examples, machined electrode 470 is sized, shaped, or otherwise configured to facilitate relatively faster and/or relatively easier fabrication of one or more of machined electrode 470 and/or assembly with other portions of a medical device system (e.g., expandable structure 190). In some examples, machined electrode 470 is sized, shaped, or otherwise configured to facilitate relatively easy attachment to expandable structure 190 (e.g., such as to reduce assembly errors, poka-yoke, or the like).

In some examples, at least a portion of machined electrode 470, including electrode body 471, is symmetric (e.g., reflectionally symmetric). As shown in FIG. 4B, machined electrode 470 is symmetric (e.g., reflectionally symmetric) about (e.g., relative to) a medial plane 479 intersecting a midpoint on an electrode face (e.g., the face of machined electrode 470 facing in the positive z direction in the example of FIG. 4A and FIG. 4B). In the example of FIG. 4B, medial plane 479 is parallel to the y-axis and extends through machined electrode 470 in the z-axis direction according to the orthogonal x-y-z axes shown in the FIG. 4B. In some examples, as shown, machined electrode 470 is symmetric such that first fixation hole 472A and second fixation hole 472B are equidistant from a medial center of machined electrode 470 (e.g., equidistance from medial plane 479). This reflectional symmetry may allow machined electrode 470 to be attached to expandable structure 190 in at least two different orientations. For example, machined electrode 470 can be attached to first projection 196A and second projection 196B of electrode attachment element 194E with either the face of machined electrode 470 in the positive z direction or the negative z direction as the leading face. Symmetry of machined electrode 470 may also facilitate balance of machined electrode 470 when fixed to one of electrode attachment element 194.

In some examples, as shown in FIG. 4B, conductor hole 474 is symmetric (e.g., reflectionally symmetric) about medial plane 479. In some examples, conductor hole 474 intersects medial plane 479. While the example of FIG. 4A and FIG. 4B depict machined electrode 470 as symmetric about medial plane 479, other configurations (including other symmetrical configurations) are contemplated.

In other examples, machined electrode 470, including electrode body 471 and/or one or more structural features of machined electrode 470, is asymmetric (e.g., asymmetric about medial plane 479). For example, machined electrode 470 can be asymmetric such that first fixation hole 472A and second fixation hole 472B are not equidistant from a medial center of machined electrode 470 (e.g., not equidistance from medial plane 479). In some examples, conductor hole 474 is not symmetric about medial plane 479 and/or does not intersect medial plane 479. In some examples, first surface 475 is asymmetric relative to medial plane 479 (e.g., first surface 475 extends relatively more in the positive x direction or the negative x direction in the example of FIG. 4A and FIG. 4B). In some examples, asymmetry of machined electrode 470 (including first surface 475) facilitates a relative larger conductive surface (e.g., first surface 475) as compared to other configurations.

FIG. 4C illustrates machined electrode 470, which may be cut from a bulk electrode 480. For example, bulk electrode 480 can be fabricated as an extruded and/or drawn component, and subsequently processed (e.g., cut, sectioned, and/or split via a suitable processes) to form one or more of individual machined electrode 470. In some examples, one or more of first fixation hole 472A, second fixation hole 472B, and/or conductor hole 474 are formed as elongated holes through bulk electrode 480 via drilling or another suitable processing, such that these holes do not have to be formed and/or defined in each individual machined electrode 470. Fabricating machined electrode 470 in this way may save time and/or facilitate a simpler, more efficient manufacturing process of many of machined electrode 470, e.g., from a single bulk electrode 480. Additionally, fabricating multiple of machined electrode 470 in this way may facilitate uniformity and/or consistency (e.g., in the shape and dimensions) between multiple of machined electrode 470.

FIG. 5A and FIG. 5B illustrate a perspective view and a side view, respectively, of a stamped electrode 570, which may be an example of one of electrodes 17 from FIG. 1 and/or one of electrodes 170 from the example of FIG. 3. Stamped electrode 570 includes an electrode body 571 that defines at least one electrically conductive surface and structural features configured to help attach stamped electrode 570 to a portion of a medical lead and/or other structure (e.g., medical lead 160 and/or expandable structure 190, as shown and described with respect to FIG. 3). For illustrative purposes, some elements described in FIG. 3 will be referenced herein with respect to stamped electrodes 570 (e.g., as corresponding mating structural features), however it should be understood that one or more of stamped electrode 570 is only one example of electrodes 170 of FIG. 3, and that other types of electrodes can additionally or alternatively be used in conjunction with the elements described in connection with FIG. 3. For example, electrode attachment element 194E, including first projection 196A and second projection 196B, as well as strut 192A of interconnected struts 192 of FIG. 3 will be referenced to illustrate how stamped electrode 570, shown in FIG. 4A and FIG. 4B, can be configured to attach to expandable structure 190 of FIG. 3.

In some examples, stamped electrode 570 includes a suitable electrically conductive material configured for transmitting and/or receiving electrical signals. In some examples, stamped electrode 570 includes one or more of titanium (Ti), tantalum (Ta), Tin (Sn), and/or a suitable combination thereof (e.g., TiTaSn and/or similar alloys). In some examples, stamped electrode 570 includes one or more of platinum (Pt) and/or Iridium (Ir) and/or a suitable combination thereof (e.g., PtIr and/or similar alloys).

As shown in the example of FIG. 5A and FIG. 5B, a portion of stamped electrode 570 defines a first surface 575, which may be an electrically conductive surface configured to transmit and/or receive electrical signals. First surface 575 can be configured to transmit and/or receive electrical signals, such as examples where stamped electrode 570 is positioned on expandable structure 190 of endovascular therapy system 100 (e.g., where endovascular therapy system 100 is configured for endovascular stimulation therapy and/or sensing a patient parameters). When affixed to expandable structure 190, first surface 575, which may be a conductive surface, is configured to face radially outward from expandable structure 190, such as in a direction radially outward from central longitudinal axis 191 of expandable structure 190 in the example of FIG. 3. In the examples of FIG. 5A and FIG. 5B, first surface 575 faces in the positive “y” direction according to the orthogonal x-y-z axes shown in FIG. 5A and FIG. 5B.

In some examples, first surface 575 includes a coating or other surface treatment to facilitate transmitting and/or receiving electrical signals. In some examples, first surface 575 includes a coating comprising one or more of Titanium Nitride (TiN), Iridium Oxide (IrOx), another suitable material, or a combination thereof. In some examples, first surface 575 additionally or alternatively includes a surface treatment, such as laser texturing to facilitate transmitting and/or receiving electrical signals. The surface treatment (e.g., laser texturing or mechanical texturing) can help effectively increase the conductive surface area of first surface 575 for a given footprint of stamped electrode 570.

As shown in the example of FIG. 5B, a portion of stamped electrode 570 defines a second surface 576, which may opposite first surface 575 (e.g., on an opposite side of electrode body 571). In some examples, an electrically insulative material 578 is applied to at least a portion of second surface 576 (e.g., all or part of second surface 576), which may reduce or inhibit transmission of electrical signals to and/or from second surface 576 of stamped electrode 570. When stamped electrode 570 is affixed to expandable structure 190, second surface 576, which may include an insulative material, is configured to face radially inward from expandable structure 190, such as in a direction radially inward toward central longitudinal axis 191 of expandable structure 190. Electrically insulative material 578 may reduce or even prevent transmission of electrical signals to and/or from less desirable regions of tissue and/or untargeted regions of tissue (e.g., a radially inward portion of a blood vessel). In this way, stamped electrode 570 can bias transmissions of electrical signals to and/or from tissue surrounding the blood vessel as compared to radially inward from the blood vessel wall. In some examples, insulative material 578 extends beyond second surface 576, such that insulative material 578 is applied to one or more sides of the electrode body 571 (sides of stamped electrode 570 different than first surface 575 and/or second surface 576).

In some examples, stamped electrode 570 is configured to bias transmissions of electrical signals to and/or from tissue surrounding the blood vessel (jugular vein 13) as compared to radially inward from the blood vessel wall. For example, in examples where stamped electrode 570 includes first surface 575 (which may be an electrically conductive surface facing radially outward towards a blood vessel wall) and second surface 576 (which may include an electrically insulative material facing radially inward from a blood vessel wall), stamped electrode 570 is configured to bias transmission of electrical signals to tissue surrounding the blood vessel in which expandable structure 190 is positioned. The biasing of transmissions of electrical signals to and/or from tissue surrounding the blood vessel can help facilitate directional electrical stimulation and/or sensing.

In some examples, at least a portion of second surface 576 is configured to interface with (e.g., contact) a strut (e.g., strut 192A) of an expandable structure (e.g., expandable structure 190). For example, when stamped electrode 570 is attached to expandable structure 190, at least a portion of second surface 576 is in contact with (e.g., rests on) a strut (e.g., strut 192A) of expandable structure 190. In some examples, at least a portion of second surface 576 defines a shape (e.g., a concave shape) that conforms to strut 192A. In some examples, a radius of curvature of at least a portion of second surface 576 is equal to or approximately equal to (e.g., to account for manufacturing tolerances) a radius of curvature of strut 192A (e.g., a radius of curvature of a cross-section of a strut 192A). In some examples, a radius of curvature of at least a portion of second surface 576 is greater than a radius of curvature of strut 192A. Configuring second surface 576 to conform to a surface of strut 192A and/or expandable structure 190 can help enable stamped electrode 570 to have a relatively low profile, which can help reduce the overall profile of expandable structure 190 and the space it occupies in a blood vessel. This may help reduce any adverse impacts on blood flow through the blood vessel.

Stamped electrode 570 can include a suitable shape, size, and/or configuration for delivering electrical stimulation therapy and/or sensing bioelectric signals from an endovascular location. In some examples, stamped electrode 570 (e.g., stamped electrode body 571) defines a non-cylindrical shape. In some examples, the non-cylindrical shape of stamped electrode 570 facilitates directional electrical stimulation therapy and/or directional sensing, such as in a direction radially outward from expandable structure 190 and/or radially outward relative to a blood vessel (e.g., jugular vein 13) and towards a blood vessel wall. For example, the non-cylindrical shape (e.g., as compared to a cylindrical electrode shape) can facilitate easier application of an insulative material to one side of stamped electrode 570 (e.g., second surface 576) while leaving another side (e.g., first surface 575) for transmitting and/or receiving electrical signals. The non-cylindrical shape of stamped electrode 570 can facilitate easier processing during manufacturing and/or assembly, such as by allowing for fixturing for application of an insulative material to only one side of stamped electrode 570 (e.g., second surface 576) and/or for processing another side of stamped electrode 570 (e.g., first surface 575) to enhance transmission of electrical signals to and/or from stamped electrode 570.

In some examples, a major surface (e.g., a major conductive surface, such as first surface 575) of stamped electrode 570 is configured to transmit and/or receive electrical signals toward a vessel wall. In some examples, first surface 575 is configured to be placed proximate (e.g., in apposition with) a vessel wall, such as to maintain contact against a vessel wall for electrical stimulation therapy and/or sensing. In some examples, at least a portion of first surface 575 is convex, and includes a radius of curvature similar to expandable structure 190 in a deployed configuration and/or similar to the blood vessel in which expandable structure 190 is configured to be placed. However, first surface 575 can define other shapes, including (but not limited to) flat, concave, irregular, and/or another shape.

In some examples, first surface 575 defines a suitable surface area for delivering stimulation therapy and/or sensing bioelectrical signals from an endovascular location, such as, but not limited to, a surface area of about 0.5 mm² to about 10 mm², such as about 1.7 mm² to about 6 mm², or overlapping ranges thereof. However, in other examples, first surface 575 defines a surface area larger or smaller than these ranges, and a suitable surface area may depend on the electrode material and/or coating applied to stamped electrode 570. For example, in some examples, first surface 575 defines a surface area of about 0.5 mm² to about 10 mm², or within a subrange thereof. In some examples, the surface area defined by one or more of stamped electrode 570 facilitates delivery of electrical stimulation therapy and/or sensing from an endovascular location while also enabling the overall form-factor of stamped electrode 570 to be relatively low-profile. For example, expandable structure 190 with one or more of stamped electrode 570 can be collapsed down to a relatively small form factor in the delivery configuration of endovascular therapy system 100 (e.g., such that expandable structure 190 and one or more of stamped electrode 570 can fit into a suitable delivery catheter).

In some examples, stamped electrode 570 is configured to receive structural features of a medical lead and/or other structures (e.g., features of medical lead 160 and/or expandable structure 190, as shown and described with respect to FIG. 3), such as for mechanically coupling one or more of stamped electrode 570 to medical lead 160 and/or another structure (e.g., expandable structure 190). The mechanical coupling of one or more of stamped electrode 570 to expandable structure 190 according to the techniques of this disclosure can help orient stamped electrode 570 with respect to the vasculatures such that first surface 575, which may be a conductive surface, faces radially outward and towards a vessel wall when expandable structure 190 with stamped electrode 570 are placed and deployed within the vasculature.

In some examples, stamped electrode 570 includes structural features configured to receive portions of an expandable structure (e.g., expandable structure 190, as shown and described with respect to FIG. 3). In the example of FIG. 5A and FIG. 5B, stamped electrode 570 (e.g., electrode body 571) defines a first fixation hole 572A and a second fixation hole 572B (collectively referred to herein as fixation holes 572), which may be configured to receive respective portions of electrode attachment elements 194, as shown and described with respect to FIG. 3. For example, in some examples, first fixation hole 572A is configured to receive first projection 196A of electrode attachment element 194E, and second fixation hole 572B is be configured to receive second projection 196B of electrode attachment element 194E. In this way, stamped electrode 570 is fixed via at least two connection points to expandable structure 190, which may minimize, or even prevent, rotation of stamped electrode 570 with respect to (e.g., about) strut 192A.

Fixation holes 572 may be sized, oriented, and otherwise configured for receiving mating portions of expandable structure 190 (e.g., first projection 196A and second projection 196B of electrode attachment element 194E). In some examples, each of first fixation hole 572A and second fixation hole 572B defines an a maximum dimension L4. Maximum dimension L4 may be a diameter in examples where first fixation hole 572A and/or second fixation hole 572B defines a circular or approximately circular shape (e.g., a circular or approximately circular cross-section in a plane defined by the x and y axes in the example of FIG. 5B). In some examples, first fixation hole 572A is spaced apart from second fixation hole 572B by a distance L6. In some examples, the distance L6 extends between center points of each of first fixation hole 572A and second fixation hole 572B. Distance L6 between first fixation hole 572A and second fixation hole 572B may correspond to respective mating structural features of electrode attachment element 194E. In some examples, distance L6 corresponds to (e.g., is equal to or nearly equal to) a distance between first projection 196A and second projection 196B of electrode attachment element 194E.

In some examples, as shown in the example of FIG. 5A and FIG. 5B, each of first fixation hole 572A and second fixation hole 572B extend entire through stamped electrode 570 (e.g., extend entirely through electrode body 571 in a direction along the z-axis according to the orthogonal x-y-z axes shown in FIG. 5A and FIG. 5B). In other examples, one or more of first fixation hole 572A and/or second fixation hole 572B extend only partially through stamped electrode 570 (e.g., only partially through electrode body 571 in a direction along the z-axis), such that first fixation hole 572A and/or second fixation hole 572B are blind holes.

In some examples, stamped electrode 570 is configured to electrically connect to one or more conductor wires, such as to facilitate electrical communication of stamped electrode 570 with a medical device (e.g., medical device 14, as shown and described in connection with FIG. 1). In some examples, a portion of stamped electrode 570 (e.g., electrode body 571) defines a conductor hole 574, where conductor hole 574 is configured to receive a conductor wire (e.g., one or more of conductor wires 166 as described with respect to FIG. 3) for electrically coupling stamped electrode 570 to a medical device. In some examples, conductor hole 574 extends entirely through stamped electrode 570 (e.g., extending entirely through electrode body 571 in a direction along the z-axis according to the orthogonal x-y-z axes shown in FIG. 5A and FIG. 5B). In other examples, conductor hole 574 extends only partially through stamped electrode 570 (e.g., only partially through electrode body 571 in a direction along the z-axis), such that conductor hole 574 is a blind hole.

In some examples, conductor hole 574 is sized to accommodate one conductor wire (e.g., of conductor wires 166). In some examples, conductor hole 574 defines a maximum dimension L5. In some examples, maximum dimension L5 is slightly larger than a width (e.g., a maximum cross-sectional dimension) of one conductor wire (e.g., of conductor wires 166). In examples where conductor hole 574 defines a circular or approximately circular shape (e.g., a circular or approximately circular cross-section in a plane defined by the x and y axes in the example of FIG. 5B), maximum dimension L5 is a diameter.

In some examples, conductor hole 574 is configured as a pass-through hole for one or more conductor wires (e.g., conductor wires 166, as described with respect to FIG. 3). Conductor hole 574 being configured as a pass-through hole can facilitate electrical connection of the conductor wire (e.g., one or more of conductor wires 166) to a second, different electrode. For example, one or more of conductor wires 166 may be fed through conductor hole 574 and electrically connected to a different (e.g., more distal) electrode. In this way, multiple of stamped electrodes 570 can be electrically connected to a common conductor of conductor wires 166 (e.g., “shorted together”), such that more than one of stamped electrode 570 can be controlled together (e.g., by a medical device, such as medical device 14 in the example of FIG. 1). In some examples, stamped electrode 570 includes more than one of conductor hole 574, such as in examples where one or more holes are configured for electrically connecting a conductor wire to stamped electrode 570 and one or more additional holes are configured as pass-through holes for routing conductor wires to at least a second, different electrode (e.g., in examples where electrodes are not “shorted” together and are controlled separately by medical device 14).

In some examples, stamped electrode 570 is sized, shaped, or otherwise configured to facilitate relatively faster and/or relatively easier fabrication of one or more of stamped electrode 570 and/or assembly with other portions of a medical device system (e.g., expandable structure 190). In some examples, stamped electrode 570 is sized, shaped, or otherwise configured to facilitate relatively easy attachment to expandable structure 190 (e.g., such as to reduce assembly errors, poka-yoke, or the like).

In some examples, at least a portion of stamped electrode 570, including electrode body 571, is symmetric (e.g., reflectionally symmetric). As shown in FIG. 5B, stamped electrode 570 is symmetric (e.g., reflectionally symmetric) about (e.g., relative to) a medial plane 579 intersecting a midpoint on an electrode face (e.g., the face of stamped electrode 570 facing in the positive z-axis direction in the example of FIG. 5A and FIG. 5B). In the example of FIG. 5B, medial plane 579 is parallel to the y-axis and extends through stamped electrode 570 in the z-axis direction according to the orthogonal x-y-z axes shown in the FIG. 5B. In some examples, stamped electrode 570 is symmetric such that first fixation hole 572A and second fixation hole 572B are equidistant from a medial center of stamped electrode 570 (e.g., equidistance from medial plane 579). This reflectional symmetry may allow stamped electrode 570 to be attached to expandable structure 190 in at least two different orientations. For example, stamped electrode 570 can be attached to first projection 196A and second projection 196B of electrode attachment element 194E with either the face of stamped electrode 570 in the positive z direction or the negative z direction as the leading face. Symmetry of stamped electrode 570 may also facilitate balance of stamped electrode 570 when fixed to one of electrode attachment element 194. While the example of FIG. 5A and FIG. 5B depict stamped electrode 570 as symmetric about medial plane 579, other configurations (including other symmetrical configurations) are contemplated.

In other examples, stamped electrode 570, including electrode body 571 and/or one or more structural features of stamped electrode 570, is asymmetric (e.g., asymmetric about medial plane 579). For example, stamped electrode 570 may be asymmetric such that first fixation hole 572A and second fixation hole 572B are not equidistant from a medial center of stamped electrode 570 (e.g., not equidistance from medial plane 579). As shown in the example of FIG. 5B, conductor hole 574 is not symmetric about medial plane 579 and/or conductor hole 574 does not intersect medial plane 579. In some examples, first surface 575 is asymmetric relative to medial plane 579 (e.g., first surface 575 extends relatively more in the positive x direction or the negative x direction in the example of FIG. 5A and FIG. 5B). In some examples, asymmetry of stamped electrode 570 (including first surface 575) facilitates a relative larger conductive surface (e.g., first surface 575) as compared to other configurations.

FIG. 5C illustrates stamped electrode 570, formed from sheet 580 (e.g., where sheet 580 includes one or more layers of electrode material from which stamped electrode 570 is formed). For example, sheet 580 can be formed (e.g., as a metallic, thin film structure) and subsequently processed (e.g., stamped and/or folded via a suitable process) to form one or more of individual stamped electrode 570. In some examples, one or more of first fixation hole 572A, second fixation hole 572B, and/or conductor hole 574 are formed during the stamping process. Fabricating stamped electrode 570 in this way may save time and/or facilitate a simpler, more efficient manufacturing process of many of stamped electrode 570, e.g., from a single sheet 580, such as compared to some examples in which a cylindrical electrode is formed by machining. Additionally, fabricating multiple of stamped electrode 570 in this way may facilitate uniformity and/or consistency (e.g., in the shape and dimensions) between multiple of stamped electrode 570.

In some examples in which stamped electrode 570 is formed from sheet 580, and in which one or more of stamped electrode 570 is subsequently attached to expandable structure 190, stamped electrode 570 is configured to deform (e.g., flex, bend, etc.) during delivery and deployment of expandable structure 190. In some examples, stamped electrode 570 is configured to exhibit elastic deformation (e.g., during transformation of expandable structure 190 between the delivery and deployed configurations and/or between the deployed and delivery configurations). For example, stamped electrode 570 may include a suitable thickness to enable some elastic (e.g., reversable) flexing or bending without permanent deformation. In some examples, sheet 580, as well as stamped electrode 570, defines a thickness of about 10 micrometers to about 100 micrometers, such as about 30 micrometers.

In some examples, electrode attachment elements 194 facilitate (e.g., accommodate) deformation or other movement of one or more of stamped electrode 570 during delivery to a target location in the vasculature and deployment to a configuration in which one or more of stamped electrode 570 are positioned in apposition with a vessel wall. For example, electrode attachment elements 194 can allow one or more of stamped electrode 570 to flex or deform around one or more of struts 192 in one or more of the delivery or deployed configuration of expandable structure 190. Such movement of stamped electrode 570, as well as some relatively minor movement relative to expandable structure 190, can help facilitate introduction of expandable structure 190 into a delivery catheter, e.g., because stamped electrode 570 can move during compression into a delivery configuration.

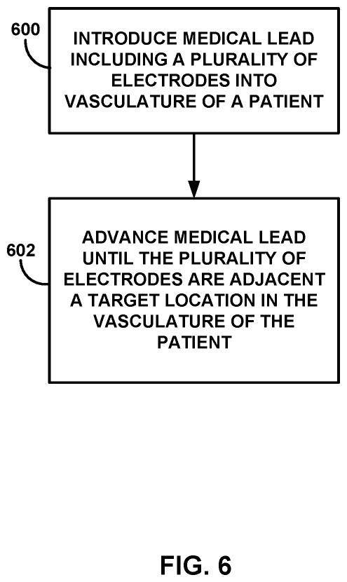

FIG. 6 is a flow diagram illustrating an example technique for using a medical device system according to the techniques of this disclosure, which may include placing a medical lead adjacent a target location in vasculature of a patient. The technique of FIG. 6 is described with respect to therapy system 10 of FIG. 1, as well as endovascular therapy system 100 of FIG. 3 (which is an example of therapy system 10 of FIG. 1), but may be used with any of the device, systems, and/or elements of systems described in this disclosure.

In the example of FIG. 6, the technique includes introducing an endovascular device (e.g., endovascular device 16 and/or medical lead 160) into vasculature of patient 12 (600). For example, a clinician may introduce at least distal portion 150 medical lead 160 through an access point in patient 12 including a femoral artery access point or radial artery access point. In some examples, one or more of an introducer sheath, a guide catheter, and/or a guidewire is used to facilitate introduction of medical lead 160 into patient 12.

In the example of FIG. 6, the technique further includes advancing medical lead 160 through the vasculature of the patient until electrodes 170 are adjacent a target location in the vasculature of patient 12 (602). In some examples, a clinician advances medical lead 160 through vasculature of patient 12 until electrodes 170 (which may include one or more of machined electrodes 470 and/or stamped electrodes 570, described in connection with FIG. 4A, FIG. 4B, FIG. 4C, FIG. 5A, FIG. 5B, FIG. 5C) are located within jugular vein 13 and positioned adjacent vagus nerve 21. Once electrodes 170 are adjacent the target location (e.g., vagus nerve 21), the clinician initiates (e.g., via programmer 20, or another suitable device) electrical stimulation therapy and/or sensing of one or more patient parameters by medical device 14.

In some examples, expandable structure 190, which can be at a distal portion of medical lead 160, is configured transform from a relatively low-profile delivery configuration to a deployed configuration in a blood vessel of a patient (e.g., within jugular vein 13 of patient 12). In some examples, expandable structure 190 remains in the delivery configuration during advancement of medical lead through the vasculature. In the delivery configuration, one or more of electrodes 170 (e.g., stamped electrodes 570) may be configured to flex and/or deform (e.g., elastically deform) to further reduce the overall profile of expandable structure with attached electrodes 170 in the delivery configuration.

In some examples, expandable structure 190 transforms to the deployed (e.g., expanded) configuration once electrodes 170 are adjacent the target site (e.g., vagus nerve 21). In the deployed configuration of expandable structure 190, one or more of electrodes 170 (e.g., machined electrodes 470 and/or stamped electrodes 570) can be positioned into apposition with the vessel wall (e.g., the vessel wall of jugular vein 13). In some examples, electrodes 170 (e.g., machined electrodes 470 and/or stamped electrodes 570) are configured to bias transmissions of electrical signals to and/or from tissue surrounding the blood vessel (jugular vein 13) as compared to radially inward from the blood vessel wall. For example, electrodes 170 can be configured with an electrically insulative material applied to a radially inward surface of each of electrode 170 (e.g., electrically insulative material 478 and/or electrically insulative material 578, as described with respect to FIG. 4B and FIG. 5B, respectively). The biasing of transmissions of electrical signals to and/or from tissue surrounding the blood vessel can help facilitate directional electrical stimulation and/or sensing. In this way, the endovascular devices described herein can help target any suitable target tissue sites (e.g., nerve structures and/or brain structures) from an endovascular location.

The techniques described in this disclosure, including those attributed to medical device 14, programmer 20, or various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium stores data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

This disclosure includes the following non-limiting examples.

Example 1: An endovascular medical device system includes an elongated body configured to be introduced in a blood vessel of a patient; an expandable structure at a distal portion of the elongated body, the expandable structure includes an expandable body portion including a plurality of interconnected struts, and a plurality of electrode attachment elements, each electrode attachment element of the plurality of electrode attachment elements including a first projection and a second projection branching off of at least one strut of the plurality of interconnected struts; and one or more electrodes coupled to the expandable structure via the plurality of electrode attachment elements, wherein each electrode of the one or more electrodes is configured to receive the first projection and the second projection of a respective electrode attachment element of the plurality of electrode attachment elements to couple the respective electrode to the expandable structure.

Example 2: The endovascular medical device system of example 1, wherein the expandable structure is configured to expand radially outwards from a relatively low-profile delivery configuration to a deployed configuration to position the one or more electrodes to deliver electrical stimulation to tissue of the patient or sense a patient parameter from a location within the blood vessel.

Example 3: The endovascular medical device system of any of examples 1 or 2, wherein each electrode of the one or more electrodes defines at least a first fixation hole and a second fixation hole, the first fixation hole configured to receive the first projection of the respective electrode attachment element and the second fixation hole configured to receive the second projection of the respective electrode attachment.

Example 4: The endovascular medical device system of any of examples 1 through 3, wherein each electrode attachment element of the plurality of electrode attachment elements is configured to minimize rotation of a given electrode of the one or more electrodes around one or more struts of the plurality interconnected struts.

Example 5: The endovascular medical device system of any of examples 1 through 4, wherein at least one electrode of the one or more electrodes defines at least one conductor hole configured to receive a conductor wire.

Example 6: The endovascular medical device system of example 5, wherein the at least one conductor hole is configured as a pass-through hole to facilitate electrical connection of the conductor wire to a second, different electrode of the one or more electrodes.

Example 7: The endovascular medical device system of any of examples 1 through 6, wherein the one or more electrodes includes one or more machined electrodes.

Example 8: The endovascular medical device system of any of examples 1 through 7, wherein the one or more electrodes includes one or more stamped electrodes.

Example 9: The endovascular medical device system of any of examples 1 through 8, wherein the one or more includes one or more machined electrodes and one or more stamped electrodes.

Example 10: The endovascular medical device system of any of examples 1 through 9, wherein at least one electrode of the one or more electrodes includes an electrically insulative material applied to an electrode surface facing radially inward toward a central longitudinal of the expandable structure.

Example 11: The endovascular medical device system of any of examples 1 through 10, wherein at least one electrode of the one or more electrodes defines an electrically conductive surface facing radially outward from a central longitudinal of the expandable structure, the electrically conductive surface defining an area of about 0.5 mm² to about 10 mm².

Example 12: The endovascular medical device system of any of examples 1 through 11, wherein the first projection extends away from a first side of the at least one strut and the second projection extends away from a second side of the at least one strut opposite the first side.

Example 13: The endovascular medical device system of any of examples 1 through 12, wherein each of the first projection and the second projection of each electrode attachment element include a portion substantially parallel to the at least one strut.

Example 14: The endovascular medical device system of any of examples 1 through 13, wherein the first projection and the second projection of each electrode attachment element are symmetric about the at least one strut of the plurality of interconnected struts.

Example 15: The endovascular medical device system of any of examples 1 through 14, wherein at least one electrode of the one or more electrodes defines a non-cylindrical shape.

Example 16: The endovascular medical device system of any of examples 1 through 15, wherein at least one electrode of the one or more electrodes is symmetric about a plane intersecting a midpoint on an electrode face.

Example 17: A method of using a medical device system includes introducing a medical device into vasculature of a patient, the medical device includes an elongated body configured to be introduced in a blood vessel of a patient; an expandable structure at a distal portion of the elongated body, the expandable structure includes an expandable body portion including a plurality interconnected struts, and a plurality of electrode attachment elements, each electrode attachment element of the plurality of electrode attachment elements including a first projection and a second projection branching off of at least one strut of the plurality of interconnected struts; and one or more electrodes coupled to the expandable structure via the plurality of electrode attachment elements, wherein each electrode of the one or more electrodes is configured to receive the first projection and the second projection of a respective electrode attachment element of the plurality of electrode attachment elements to couple the respective electrode to the expandable structure; and advancing the medical device until the one or more electrodes are at or near a target location in the vasculature of the patient.

Example 18: The method of example 17, wherein the expandable structure is configured to expand radially outwards from a relatively low-profile delivery configuration to a deployed configuration to position the one or more electrodes to deliver electrical stimulation to tissue of the patient or sense a patient parameter from a location within the blood vessel.

Example 19: The method of any of examples 17 or 18, wherein each electrode of the one or more electrodes defines at least a first fixation hole and a second fixation hole, the first fixation hole configured to receive the first projection of the respective electrode attachment element and second fixation hole configured to receive the second projection of the respective electrode attachment.

Example 20: The method of any of examples 17 through 19, wherein each electrode attachment element of the plurality of electrode attachment elements is configured to minimize rotation of a given electrode of the one or more electrodes around one or more struts of the plurality interconnected struts.

Example 21: The method of any of examples 17 through 20, wherein at least one electrode of the one or more electrodes defines at least one conductor hole configured to receive a conductor wire.

Example 22: The method of example 21, wherein the at least one conductor hole is configured as a pass-through hole to facilitate electrical connection of the conductor wire to a second, different electrode of the one or more electrodes.

Example 23: The method of any of examples 17 through 22, wherein the one or more electrodes includes one or more machined electrodes.

Example 24: The method of any of examples 17 through 23, wherein the one or more electrodes includes one or more stamped electrodes.

Example 25: The method of any of examples 17 through 24, wherein the one or more electrodes includes one or more machined electrodes and one or more stamped electrodes.

Example 26: The method of any of examples 17 through 25, wherein at least one electrode of the one or more electrodes includes an electrically insulative material applied to an electrode surface facing radially inward toward a central longitudinal of the expandable structure.

Example 27: The method of any of examples 17 through 26, wherein at least one electrode of the one or more electrodes defines an electrically conductive surface facing radially outward from a central longitudinal of the expandable structure, the electrically conductive surface defining an area an area of about 0.5 mm² to about 10 mm².

Example 28: The method of any of examples 17 through 27, wherein the first projection extends away from a first side of the at least one strut and the second projection extends away from a second side of the at least one strut opposite the first side.

Example 29: The method of any of examples 17 through 28, wherein each of the first projection and the second projection of each electrode attachment element include a portion substantially parallel to the at least one strut.

Example 30: The method of any of examples 17 through 29, wherein the first projection and the second projection of each electrode attachment element are symmetric about the at least one strut of the plurality of interconnected struts.

Example 31: The method of any of examples 17 through 30, wherein at least one electrode of the one or more electrodes defines a non-cylindrical shape.

Example 32: The method of any of examples 17 through 31, wherein at least one electrode of the one or more electrodes is symmetric about a plane intersecting a midpoint on an electrode face.

Example 33: An electrode includes an electrode body defining: a first fixation hole configured to receive a first projection of an expandable structure; a second fixation hole separate from the first fixation hole and configured to receive a second projection of the expandable structure; and at least one conductor hole configured to receive a conductor wire; wherein the electrode body includes a first electrode surface, the first electrode surface being electrically conductive, wherein the electrode body includes a second electrode surface opposite the first electrode surface with an electrically insulative material applied to the second electrode surface, and wherein the electrode body defines a non-cylindrical shape.

Example 34: The electrode of example 33, wherein the second electrode surface defines an area of about 0.5 mm² to about 10 mm².

Example 35: The electrode of any of examples 33 or 34, wherein the electrode body is symmetric about a plane intersecting a midpoint on an electrode face.

Example 36: The electrode of any of examples 33 or 34, wherein the electrode body is asymmetric about a plane intersecting a midpoint on an electrode face.

Example 37: The electrode of any of examples 33 through 36, wherein the at least one conductor hole is configured as a pass-through hole to facilitate electrical connection of the conductor wire to a different electrode.

Various examples of the disclosure have been described. Any combination of the described systems, operations, or functions is contemplated. These and other examples are within the scope of the following claims.

What is claimed is:

1. An endovascular medical device system comprising:
an elongated body configured to be introduced in a blood vessel of a patient;
an expandable structure at a distal portion of the elongated body, the expandable structure comprising:
an expandable body portion including a plurality of interconnected struts, and
a plurality of electrode attachment elements, each electrode attachment element of the plurality of electrode attachment elements including a first projection and a second projection branching off of at least one strut of the plurality of interconnected struts; and
one or more electrodes coupled to the expandable structure via the plurality of electrode attachment elements,
wherein each electrode of the one or more electrodes is configured to receive the first projection and the second projection of a respective electrode attachment element of the plurality of electrode attachment elements to couple the respective electrode to the expandable structure.

2. The endovascular medical device system of claim 1, wherein the expandable structure is configured to expand radially outwards from a relatively low-profile delivery configuration to a deployed configuration to position the one or more electrodes to deliver electrical stimulation to tissue of the patient or sense a patient parameter from a location within the blood vessel.

3. The endovascular medical device system of claim 1, wherein each electrode of the one or more electrodes defines at least a first fixation hole and a second fixation hole, the first fixation hole configured to receive the first projection of the respective electrode attachment element and the second fixation hole configured to receive the second projection of the respective electrode attachment.

4. The endovascular medical device system of claim 1, wherein at least one electrode of the one or more electrodes defines at least one conductor hole configured to receive a conductor wire.

5. The endovascular medical device system of claim 4, wherein the at least one conductor hole is configured as a pass-through hole to facilitate electrical connection of the conductor wire to a second, different electrode of the one or more electrodes.

6. The endovascular medical device system of claim 1, wherein the one or more electrodes includes one or more machined electrodes.

7. The endovascular medical device system of claim 1, wherein the one or more electrodes includes one or more stamped electrodes.

8. The endovascular medical device system of claim 1, wherein the one or more includes one or more machined electrodes and one or more stamped electrodes.

9. The endovascular medical device system of claim 1, wherein at least one electrode of the one or more electrodes includes an electrically insulative material applied to an electrode surface facing radially inward toward a central longitudinal of the expandable structure.

10. The endovascular medical device system of claim 1, wherein at least one electrode of the one or more electrodes defines an electrically conductive surface facing radially outward from a central longitudinal of the expandable structure, the electrically conductive surface defining an area of about 0.5 mm² to about 10 mm².

11. The endovascular medical device system of claim 1, wherein the first projection extends away from a first side of the at least one strut and the second projection extends away from a second side of the at least one strut opposite the first side.

12. A method of using a medical device system, the method comprising:
introducing a medical device into vasculature of a patient, the medical device comprising:
an elongated body configured to be introduced in a blood vessel of a patient;
an expandable structure at a distal portion of the elongated body, the expandable structure comprising:
an expandable body portion including a plurality interconnected struts, and
a plurality of electrode attachment elements, each electrode attachment element of the plurality of electrode attachment elements including a first projection and a second projection branching off of at least one strut of the plurality of interconnected struts; and
one or more electrodes coupled to the expandable structure via the plurality of electrode attachment elements,
wherein each electrode of the one or more electrodes is configured to receive the first projection and the second projection of a respective electrode attachment element of the plurality of electrode attachment elements to couple the respective electrode to the expandable structure; and
advancing the medical device until the one or more electrodes are at or near a target location in the vasculature of the patient.

13. The method of claim 12, wherein the expandable structure is configured to expand radially outwards from a relatively low-profile delivery configuration to a deployed configuration to position the one or more electrodes to deliver electrical stimulation to tissue of the patient or sense a patient parameter from a location within the blood vessel.

14. The method of claim 12, wherein each electrode of the one or more electrodes defines at least a first fixation hole and a second fixation hole, the first fixation hole configured to receive the first projection of the respective electrode attachment element and second fixation hole configured to receive the second projection of the respective electrode attachment.

15. The method of claim 12, wherein at least one electrode of the one or more electrodes defines at least one conductor hole configured to receive a conductor wire.

16. The method of claim 15, wherein the at least one conductor hole is configured as a pass-through hole to facilitate electrical connection of the conductor wire to a second, different electrode of the one or more electrodes.

17. The method of claim 12, wherein the one or more electrodes includes one or more machined electrodes.

18. The method of claim 12, wherein the one or more electrodes includes one or more stamped electrodes.

19. The method of claim 12, wherein the one or more electrodes includes one or more machined electrodes and one or more stamped electrodes.

20. The method of claim 12, wherein at least one electrode of the one or more electrodes includes an electrically insulative material applied to an electrode surface facing radially inward toward a central longitudinal of the expandable structure.

21. The method of claim 12, wherein at least one electrode of the one or more electrodes defines an electrically conductive surface facing radially outward from a central longitudinal of the expandable structure, the electrically conductive surface defining an area of about 0.5 mm² to about 10 mm².

22. The method of claim 12, wherein the first projection extends away from a first side of the at least one strut and the second projection extends away from a second side of the at least one strut opposite the first side.

23. An electrode comprising:
an electrode body defining:
a first fixation hole configured to receive a first projection of an expandable structure;
a second fixation hole separate from the first fixation hole and configured to receive a second projection of the expandable structure; and
at least one conductor hole configured to receive a conductor wire;
wherein the electrode body includes a first electrode surface, the first electrode surface being electrically conductive,
wherein the electrode body includes a second electrode surface opposite the first electrode surface with an electrically insulative material applied to the second electrode surface, and
wherein the electrode body defines a non-cylindrical shape.

24. The electrode of claim 23, wherein the at least one conductor hole is configured as a pass-through hole to facilitate electrical connection of the conductor wire to a different electrode.
